(19) United States
(12) Patent Application Publication
(10) Pub. No.: US 2026/0174877 A1
(43) Pub. Date: Jun. 25, 2026

(54) COMPOUNDS AND METHODS FOR THE TREATMENT OF DERMAL AND OCULAR DISORDERS

(71) Applicant: Azura Ophthalmics Ltd., Tel Aviv (IL)

(72) Inventors: Marc GLEESON, Longueville (AU); Charles BOSWORTH, Las Vegas, NV (US)

(21) Appl. No.: 19/426,759

(22) Filed: Dec. 19, 2025

Related U.S. Application Data
(60) Provisional application No. 63/736,343, filed on Dec. 19, 2024.

(51) Int. Cl. A61K 47/54

(52) U.S. Cl. CPC A61K 47/545; A61K 47/549

(57) ABSTRACT

Described herein are compositions and methods for the treatment or prevention of dermal or ocular surface disorders, such as those associated with bacterial and/or parasitic infections. Bacterial and/or parasitic infections include mites (e.g., demodex), worms, protozoa (e.g., amoeba (e.g., acanthamoeba or cryptosporidiosis)), onchocerciasis, lice, scabies, nematodes, or chlamydia. Said compositions and methods comprise compounds which demonstrate immunological, keratolytic, anti-histamine, anti-inflammatory, and/or other desirable activities. Topical administration of said compositions to the eyelid margin or surrounding areas provides therapeutic benefit to patients suffering from ocular surface disorders.

### CROSS-REFERENCE

This application claims the benefit of U.S. Provisional Application No. 63/736,343, filed Dec. 19, 2024, which is incorporated herein by reference in its entirety.

### BACKGROUND OF THE INVENTION

Dry eye disease (DED) is reported to have a global prevalence of 5 to 50%. While there are some treatments available for treating DED, DED is generally a symptom of a variety of underlying diseases, so effective treatments for individual patients can remain elusive.

### SUMMARY OF THE INVENTION

Provided in certain embodiments herein are compounds, such as compounds suitable for (e.g., simultaneously) targeting multiple underlying etiologies of symptomatic disease, such as dry eye disease (DED). In some instances, such compounds are suitable for treating multiple underlying etiologies of disease, which can be useful in providing (1) better outcomes for individuals suffering from disease as a result of multiple etiologies, and (2) improving disease response in a class of patients who may have different causes of disease. Also provided in certain embodiments herein are pharmaceutical (e.g., dermal and/or ophthalmic) compositions comprising such compounds, and methods of treating disease by administering a compound or composition provided herein to an individual (e.g., an individual suffering from such a disease).

In specific embodiments, the disease treated by any method provided herein is an ocular, periocular, or dermal disorder, such as dry eye disease (DED). In specific embodiments, the disease treated by any method provided herein is associated with an infection, such as a demodex infestation or infection.

In some instances, provided herein is a compound that comprises a radical of an antiparasitic agent described herein (e.g., moxidectin or ivermectin). In some instances, provided herein is a compound that comprises a radical of an antiparasitic agent described herein (e.g., moxidectin or ivermectin) and a radical of a keratolytic agent described herein. In some instances, provided herein is a compound that comprises a radical of an antiparasitic agent described herein (e.g., moxidectin or ivermectin) and a radical of a H1 antagonist described herein. In some embodiments, provided herein is a compound having a structure represented by Formula (X): Y-L-Z, wherein Y is an antiparasitic agent described herein, L is a linker described herein, and Z is a radical of a keratolytic agent described herein or a radical of a H1 antagonist described herein.

In some instances, provided herein is a compound that delivers a therapeutically effective amount of (e.g., a free form of) an anti-parasitic agent, such as an anti-parasitic agent described herein (e.g., where the anti-parasitic agent reduces the amount of a demodex mites and/or reduces or eliminates the infestation thereof) and/or (e.g., a free form of) an H1 antagonist, such as an H1 antagonist described herein (e.g., where the H1 antagonist reduces release of histamine, such as providing relief of a redness, itching, hypersensitivity, or the like). In some instances, the free form of the antiparasitic agent is ivermectin. In some instances, the free form of the antiparasitic agent is moxidectin. In some instances, the free form of the H1 antagonist is selected from the group consisting of olopatadine, cetirizine, acrivastine, bilastine, fexofenadine, levocabastine, hydroxyzine, periciazine, and quetiapine.

In some instances, provided herein is a compound that delivers a therapeutically effective amount of (e.g., a free form of) an anti-parasitic agent, such as an anti-parasitic agent described herein (e.g., where the anti-parasitic agent reduces the amount of a demodex mites and/or reduces or eliminates the infestation thereof) and/or (e.g., a free form of) at least one keratolytic agent, such as a keratolytic agent described herein (e.g., where the keratolytic agent breaks down, reduces, and/or eliminates keratinized material). In some instances, the free form of the antiparasitic agent is ivermectin. In some instances, the free form of the antiparasitic agent is moxidectin.

Provided in some embodiments herein is a compound having a structure represented by Formula (X):

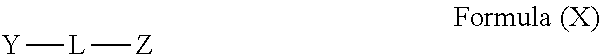

wherein:
   Y is a radical of an anti-parasitic agent;
   Z is a radical of a keratolytic agent or a radical of an H1 antagonist; and
   L is a linker (e.g., a bond).

In some embodiments, Y has a structure represented by Formula (1-A):

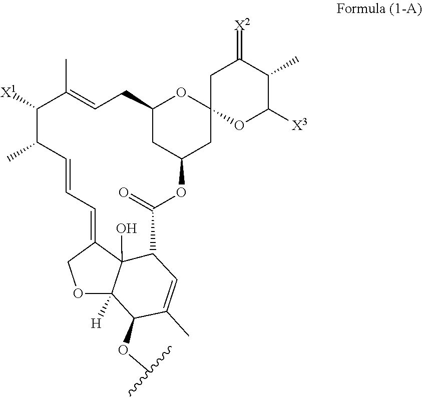

wherein,
X¹ is absent or

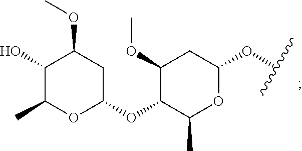

X² is absent or ═NOCH₃; and
X³ is substituted or unsubstituted (e.g., branched) alkyl;
or a pharmaceutically acceptable salt or solvate thereof.

In some embodiments, X¹ is absent. In some embodiments, X² is ═NOCH₃. In some embodiments, X³ is branched alkyl. In some embodiments, X³ is —C(CH₃)═CHCH(CH₃)₂.

In some embodiments, Y is a radical of moxidectin.

In some embodiments, Y is not a radical of ivermectin or metronidazole. In some embodiments, Y is not a radical of ivermectin. In some embodiments, Y is not a radical of metronidazole.

In some embodiments, Y does not have the following structure:

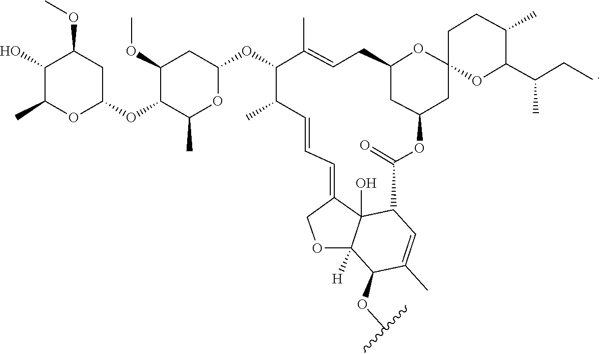

In some embodiments, Y does not have the following structure:

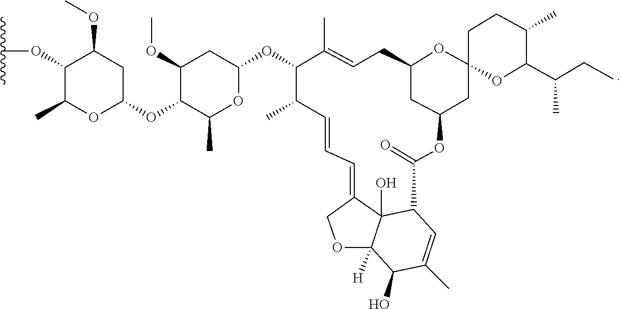

In some embodiments, Z is a radical of a keratolytic agent, such as a radical of a keratolytic agent described herein. In some embodiments, Z is a radical of a H1 antagonist, such as a radical of a H1 antagonist described herein.

Provided in some embodiments herein is a compound having a structure represented by Formula (A-1):

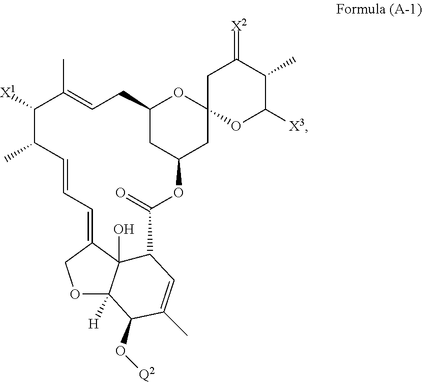

wherein,
   X¹ is absent or

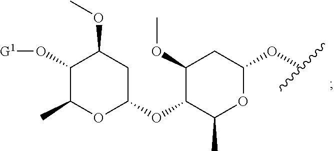

X² is absent or ═NOCH₃;
X³ is substituted or unsubstituted (e.g., branched) alkyl;
G¹, if present, is hydrogen, substituted or unsubstituted alkyl, or -L¹-D¹;
G² is hydrogen, substituted or unsubstituted alkyl, or -L² D²;
   at least one of G¹ or G² being -L¹-D¹ or -L²-D²;
   D¹ and D² are each independently a radical of an H1 antagonist; and
   L¹ and L² are each independently a linker;
or a pharmaceutically acceptable salt or solvate thereof.

In some embodiments, X¹ is absent.

In some embodiments, X² is ═NOCH₃.

In some embodiments, X³ is branched alkyl. In some embodiments, X³ is —C(CH₃)═CHCH(CH₃)₂.

In some embodiments, G² is -L²-D².

In some embodiments, the compound does not have a structure represented by Formula (I):

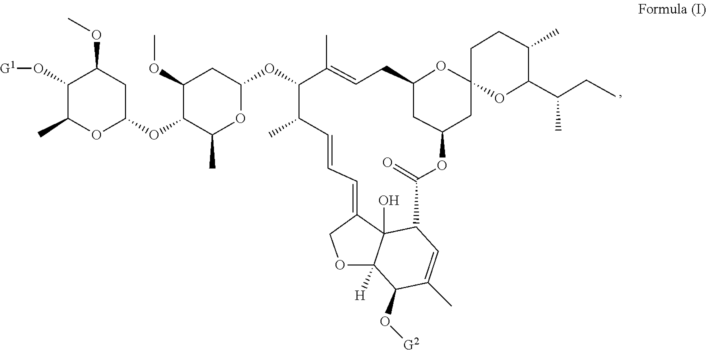

wherein,
   G¹ is hydrogen, substituted or unsubstituted alkyl, or -L¹-D¹;
   G² is hydrogen, substituted or unsubstituted alkyl, or -L²-D²;
      at least one of G¹ or G² being -L¹-D¹ or -L²-D²;
      D¹ and D² are each independently a radical of an H1 antagonist; and
      L¹ and L² are each independently a linker;
or a pharmaceutically acceptable salt or solvate thereof.

Provided in some embodiments herein is a compound having a structure represented by Formula (A-2):

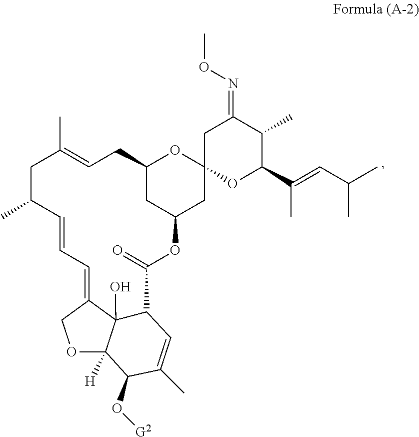

wherein,
   G² is -L²-D²;
      D² is a radical of an H1 antagonist; and
      L² is a linker;
or a pharmaceutically acceptable salt or solvate thereof.

In some embodiments, the H1 antagonist is a first-generation H1 antagonist. In some embodiments, the H1 antagonist is a second-generation H1 antagonist. In some embodiments, the H1 antagonist is selected from the group consisting of olopatadine, cetirizine, acrivastine, bilastine, fexofenadine, levocabastine, hydroxyzine, periciazine, and quetiapine.

Provided in some embodiments herein is a compound having a structure represented by Formula (B-1):

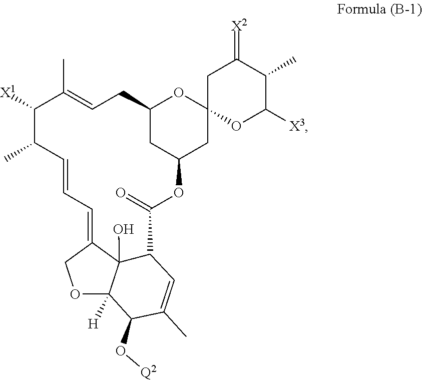

wherein,
   X¹ is absent or

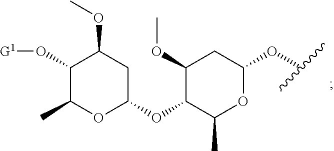

X² is absent or ═NOCH₃;
X³ is substituted or unsubstituted (e.g., branched) alkyl;
Q¹, if present, is hydrogen, substituted or unsubstituted alkyl, or -L^{A}-R¹;
Q² is hydrogen, substituted or unsubstituted alkyl, or -L^{B}-R²;
   at least one of Q¹ or Q² being -L^{A}-R¹ or -L^{B}-R²;
   R¹ and R² are each independently a radical of a keratolytic agent; and
   L^{A} and L^{B} are each independently a linker;
or a pharmaceutically acceptable salt or solvate thereof.

In some embodiments, X¹ is absent.

In some embodiments, X² is ═NOCH₃.

In some embodiments, X³ is branched alkyl. In some embodiments, X³ is —C(CH₃)═CHCH(CH₃)₂.

In some embodiments, Q² is -L^{B}-R².

In some embodiments, the compound does not have a structure represented by Formula (II):

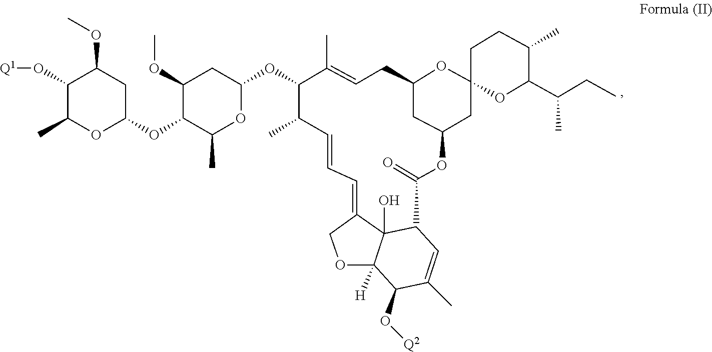

wherein,
   Q¹ is hydrogen, substituted or unsubstituted alkyl, or -L^{A}-R¹;
   Q² is hydrogen substituted or unsubstituted alkyl, or -L^{B}-R²;
      at least one of Q¹ or Q² being -L^{A}-R¹ or -L^{B}-R²;
      R¹ and R² are each independently a radical of a keratolytic agent; and
      L^{A} and L^{B} are each independently a linker;
or a pharmaceutically acceptable salt or solvate thereof.

Provided in some embodiments herein is compound having a structure represented by Formula (B-2):

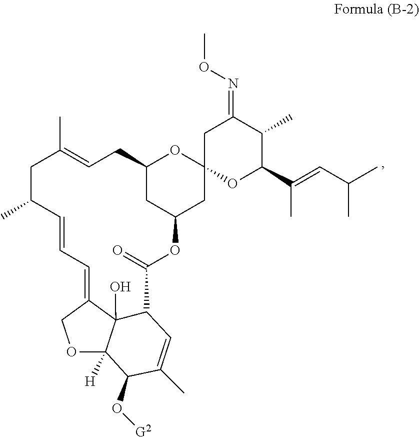

wherein,
Q² is -L^{B}-R²;
   R² is a radical of a keratolytic agent; and
   L^{B} is a linker;
or a pharmaceutically acceptable salt or solvate thereof.

In some embodiments, each radical of a keratolytic agent (e.g., Z or R²) comprises one or more keratolytic group.

In some embodiments, each radical of a keratolytic agent (e.g., Z or R²) comprises one or more group, each group being independently selected from the group consisting of —O—, oxo, substituted or unsubstituted (e.g., branched or straight) alkyl (alkylenyl), substituted or unsubstituted (e.g., branched or straight) heteroalkyl (heteroalkylenyl), substituted or unsubstituted alkoxyl, substituted or unsubstituted cycloalkyl, and substituted or unsubstituted heterocyclyl.

In some embodiments, each radical of a keratolytic agent (e.g., Z or R²) is substituted or unsubstituted (e.g., branched or straight) alkyl (alkylenyl) or substituted or unsubstituted (e.g., branched or straight) heteroalkyl (heteroalkylenyl). In some embodiments, each radical of a keratolytic agent (e.g., Z or R²) is (e.g., branched or straight) alkyl (alkylenyl) substituted with one or more substituent, each substituent being independently selected from the group consisting of oxo, hydroxy, alkyl, and substituted or unsubstituted heterocyclyl. In some embodiments, each radical of a keratolytic agent (e.g., Z or R²) is (e.g., branched or straight) heteroalkyl (heteroalkylenyl) substituted with one or more substituent, each substituent being independently selected from the group consisting of oxo, alkyl, thiol, thioalkyl, and substituted or unsubstituted heterocyclyl.

In some embodiments, each radical of a keratolytic agent (e.g., Z or R²) has a structure represented by:

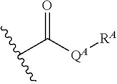

wherein:
   Q^{A} is —O— or —(CR^{B}R^{C})ₘ;
   m is 1-6;
   each R^{B} and R^{C} is independently H, halo, alkyl, alkoxy, haloalkyl, or thioalkyl;
      or an adjacent R^{B} and R^{C} combine to the atoms to which they are attached to form an oxo; and
   R^{A} is alkyl, heteroalkyl, heterocyclyl, alkoxy, or hydroxy, the alkyl, heteroalkyl, heterocyclyl, or alkoxy each independently being optionally substituted.

In some embodiments, Q^{A} is O.

In some embodiments, —(CR^{B}R^{C})ₘ—.

In some embodiments, each R^{B} and R^{C} is independently H or C₁-C₆ alkyl. In some embodiments, each R^{B} and R^{C} is independently H or CH₃.

In some embodiments, m is 1-4.

In some embodiments, R^{A} is substituted (e.g., branched or linear) heteroalkyl (e.g., branched heteroalkyl substituted with one or more oxo and/or substituted or unsubstituted heterocyclyl (e.g., dithiolanyl or dithiolanyl oxide)).

In some embodiments, RA is substituted (e.g., branched or linear) alkyl (e.g., alkyl substituted with one or more oxo, hydroxy, and/or substituted or unsubstituted heterocyclyl (e.g., dithiolanyl or dithiolanyl oxide)).

In some embodiments, R^{A} is optionally substituted heterocyclyl (e.g., dithiolanyl or dithiolanyl oxide).

In some embodiments, Q^{A} is O and R is unsubstituted alkyl or substituted or unsubstituted heteroalkyl (e.g., heteroalkyl substituted with oxo and/or optionally substituted heterocyclyl (e.g., dithiolanyl or dithiolanyl oxide)).

In some embodiments, Q^{A} is —(CR^{B}R^{C})ₘ—, m is 1-4, R^{B} and R^{C} are each independently H or C₁-C₆ alkyl, and R^{A} is optionally substituted heterocyclyl, alkyl substituted with optionally substituted heterocyclyl, alkyl substituted with one or more oxo and hydroxy, heteroalkyl substituted with optionally substituted heterocyclyl, or heteroalkyl substituted with one or more oxo. In some embodiments, the optionally substituted heterocyclyl is dithiolanyl or dithiolanyl oxide. In some embodiments, the optionally substituted heterocyclyl is:

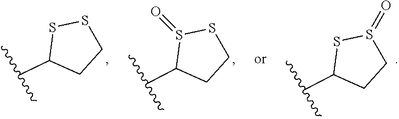 [SMILES: *C1CCSS1 ; *C1CCSS1=O ; *C1CCS(=O)S1]

In some embodiments, R^{A} is —CH₃, —CH(CH₃)₂, substituted C₁-C₆ alkyl (e.g., alkyl substituted with CH₃, oxo, hydroxy, and/or dithiolanyl or dithiolanyl oxide), or substituted C₁-C₆ heteroalkyl (e.g., heteroalkyl substituted with CH₃, oxo, and/or dithiolanyl or dithiolanyl oxide).

In some embodiments, each linker (e.g., L¹, L², L^{A} or L^{B}) is independently a bond, substituted or unsubstituted (e.g., branched or straight) alkyl (alkylenyl), or substituted or unsubstituted (e.g., branched or straight) heteroalkyl (heteroalkylenyl).

In some embodiments, each linker (e.g., L¹, L², L^{A} or L^{B}) is independently a bond or substituted or unsubstituted (e.g., branched or straight) alkyl (alkylenyl).

In some embodiments, each linker (e.g., L¹, L², L^{A} or L^{B}) is independently a bond, >C(═O), —C(═O)O—, —C(═O)OCH(CH₃)—, —CH(CH₃)—, or —CH₂—.

In some embodiments, the linker (e.g., L¹, L², L^{A} or L^{B}) of a compound described herein is a bond.

In some embodiments, the linker of a compound described herein is a non-hydrolyzable linker.

In some embodiments, the linker of a compound described herein is a hydrolyzable linker.

In some embodiments, a compound described herein (e.g., the linker of a compound described herein) is enzymatically stable. In some embodiments, a compound described herein (e.g., the linker of a compound described herein) is not cleavable by enzymes, such as enzymes of the skin and/or eye (e.g., esterases, hydrolases, or reductases).

In some embodiments, a compound described herein (e.g., the linker of a compound described herein) is stable in an aqueous environment, such as a biological environment and/or a buffered solution).

In some embodiments, a compound described herein has anti-parasitic activity.

In some embodiments, a compound described herein has keratolytic activity.

In some embodiments, a compound described herein has antihistamine activity.

Provided in some embodiments herein is a topical pharmaceutical composition (e.g., peridermal or periocular) comprising a compound having a structure described herein, such as a structure represented by Formula (X), Formula (1-A), Formula (A-1), Formula (A-2), Formula (B-1), or Formula (B-2).

In some embodiments, provided herein is a pharmaceutical composition comprising any compound provided herein, such as a compound represented by any structure herein, such as, for example, Formula (X), Formula (1-A), Formula (A-1), Formula (A-2), Formula (B-1), or Formula (B-2), or a pharmaceutically acceptable salt thereof, and at least one pharmaceutically acceptable excipient. In some embodiments, the pharmaceutical composition is suitable for topical administration (e.g., to the skin and/or (e.g., in or around) the eye). In some embodiments, the pharmaceutical composition is suitable for ophthalmic administration. In some embodiments, the pharmaceutical composition is suitable for topical ophthalmic administration. In some embodiments, (e.g., topical) ophthalmic administration is administration in and/or around the eye.

In some embodiments, the pharmaceutical composition is suitable for dermal administration.

In some embodiments, a compound or a pharmaceutical composition comprising any compound provided herein, such as a compound of any one of Formula (X), Formula (1-A), Formula (A-1), Formula (A-2), Formula (B-1), or Formula (B-2), or a pharmaceutically acceptable salt thereof, is substantially hydrolytically stable (e.g., stable in an aqueous composition (e.g., solution), such as a buffer solution or ophthalmically-acceptable (aqueous) composition). In some embodiments, the compound or the pharmaceutical composition is formulated in an aqueous vehicle. In some embodiments, the compound or the pharmaceutical composition is formulated and stored in an aqueous vehicle. In some instances, compositions or formulations provided herein are chemically and/or physically stable in an aqueous composition.

In some embodiments, a compound or a pharmaceutical composition comprising any compound provided herein, such as a compound of any one of Formula (X), Formula (1-A), Formula (A-1), Formula (A-2), Formula (B-1), or Formula (B-2), or a pharmaceutically acceptable salt thereof, is substantially enzymatically stable (e.g., stable in a biological environment, such as skin or in or around an ocular surface. In some instances, compositions or formulations provided herein are chemically and/or physically stable in a biological environment.

In some embodiments, a compound provided herein, such as a compound of any one of Formula (X), Formula (1-A), Formula (A-1), Formula (A-2), Formula (B-1), or Formula (B-2), or a pharmaceutically acceptable salt thereof, is reduced to one or more keratolytic agent (e.g., a free form of a radical of Formula (X), Formula (1-A), Formula (A-1), Formula (A-2), Formula (B-1), or Formula (B-2), such as wherein R is a negative charge or H) and/or hydrolyzed to an active pharmaceutical agent (e.g., a free form of a radical of Formula (X), Formula (1-A), Formula (A-1), Formula (A-2), Formula (B-1), or Formula (B-2), such as wherein R is a negative charge or H). In some embodiments, the compound or pharmaceutical composition is reduced to one or more keratolytic agent in an ocular space. In some embodiments, the compound or pharmaceutical composition is reduced to one or more keratolytic agent by a reductase in an ocular space.

In some embodiments, a compound provided herein, such as a compound of any one of Formula (X), Formula (1-A), Formula (A-1), Formula (A-2), Formula (B-1), or Formula (B-2), or a pharmaceutically acceptable salt thereof, is hydrolyzed to an active pharmaceutical agent (e.g., a free form of a radical of Formula (X), Formula (1-A), Formula (A-1), Formula (A-2), Formula (B-1), or Formula (B-2), such as wherein R is a negative charge or H) and a keratolytic agent. In some embodiments, the compound or pharmaceutical composition is hydrolyzed to an active pharmaceutical agent and a keratolytic agent in an ocular space. In some embodiments, the compound or pharmaceutical composition is hydrolyzed to an active pharmaceutical agent and a keratolytic agent by an esterase in an ocular space.

In some embodiments, the active pharmaceutical agent is an anti-parasitic and/or an antihistamine. In some embodiments, the active pharmaceutical agent is an anti-parasitic and/or an anti-inflammatory agent. In some embodiments, the active pharmaceutical agent is an anti-parasitic. In some embodiments, the active pharmaceutical agent is an antihistamine. In some embodiments, the active pharmaceutical agent is an anti-inflammatory agent. In some embodiments the anti-parasitic, antihistamine, and/or anti-inflammatory agent is ivermectin, moxidectin, olopatadine, cetirizine, acrivastine, bilastine, fexofenadine, levocabastine, hydroxyzine, periciazine, or quetiapine. In some embodiments the anti-parasitic, antihistamine, and/or anti-inflammatory agent is ivermectin, olopatadine, cetirizine, acrivastine, bilastine, fexofenadine, levocabastine, hydroxyzine, periciazine, or quetiapine. In some embodiments the anti-parasitic, antihistamine, and/or anti-inflammatory agent is moxidectin, olopatadine, cetirizine, acrivastine, bilastine, fexofenadine, levocabastine, hydroxyzine, periciazine, or quetiapine. In some embodiments the anti-parasitic is ivermectin. In some embodiments the anti-parasitic is moxidectin. In some embodiments the antihistamine is olopatadine, cetirizine, acrivastine, bilastine, fexofenadine, levocabastine, hydroxyzine, periciazine, or quetiapine. In some embodiments, the active keratolytic agent comprises a carboxylic acid, a thiol (—SH), and/or a thioether (S—S). In some embodiments, the keratolytic agent is or comprises a carboxylic acid. In some embodiments, the carboxylic acid is selected from the group consisting of acetic acid, glycolic acid, lactic acid, lipoic acid, pivalic acid, isobutryic acid, butyric acid, propionic acid, formic acid, and carbonic acid. In some embodiments, the active keratolytic agent is or comprises a thiol (—SH). In some embodiments, the active keratolytic agent is or comprises a thioether (S—S).

Described in some embodiments herein is a pharmaceutical composition comprising any compound provided herein, such as a compound of any one of Formula (X), Formula (1-A), Formula (A-1), Formula (A-2), Formula (B-1), or Formula (B-2), or a pharmaceutically acceptable salt thereof. In certain embodiments, the composition further comprises an amount of a free form of a radical of any of Formula (X), Formula (1-A), Formula (A-1), Formula (A-2), Formula (B-1), or Formula (B-2), or the like (such as wherein the free form is the radical, wherein R is a negative charge or an H). In some embodiments, a composition provided herein comprises a (e.g., weight or molar) ratio of a compound provided herein to a free form of a radical of Formula (X), Formula (1-A), Formula (A-1), Formula (A-2), Formula (B-1), or Formula (B-2), or a pharmaceutically acceptable salt thereof (e.g., wherein R is a negative charge or an H) of about 1:99 to about 100:0 (e.g., the amount of the free form of the radical relative to the overall amount of free form of the radical plus the conjugate is between 0% (weight or molar) and 99%). In some embodiments, the relative amount of the free form of the radical is 0% to about 50%, such 0% to about 20%, 0% to about 10%, about 0.1% to about 10%, about 0.1% to about 5%, less than 5%, less than 2.5%, less than 2%, or the like (percentages being weight/weight or mole/mole percentages). In some instances, such aqueous compositions are pre-manufactured or are manufactured at the time of application in order to maintain high concentrations of the compound relative to the free form of a radical thereof. In some embodiments, such concentrations of the compound are present in the composition for at least 45 minutes in an aqueous composition (such as in an aqueous composition, e.g., a HEPES buffer, such as under the conditions described herein). Further, in some instances, compounds provided herein release free form of a radical of a compound of Formula (X), Formula (1-A), Formula (A-1), Formula (A-2), Formula (B-1), or Formula (B-2) (e.g., wherein R is a negative charge or H), such as when administered to an individual (e.g., ocular (e.g., peri-ocular) or dermatological administration). In more specific instances, when administered to an individual at a location with esterases and/or reductases present, rapid release of active (free) forms of a radical of Formula (X), Formula (1-A), Formula (A-1), Formula (A-2), Formula (B-1), or Formula (B-2), (e.g., wherein R is a negative charge or H) (and, a keratolytic agent and/or agent that further produces active keratolytic agent(s) (e.g., by further hydrolysis and/or reduction thereof)).

In some instances, compounds provided herein are enzymatically stable. In some instances, compounds provided herein are not cleavable by enzymes, such as enzymes of the skin and/or eye (e.g., esterases, hydrolases, or reductases). In some instances, compounds provided herein are hydrolytically stable. In some instances, compounds provided herein are enzymatically and hydrolytically stable. In some instances, compounds provided herein are stable in an aqueous environment, such as a biological environment and/or a buffered solution). In some instances, compounds provided herein have anti-parasitic activity. In some instances, compounds provided herein have keratolytic activity.

In some embodiments, provided herein a compound or a pharmaceutical composition comprising any compound provided herein, such as a compound of any one of Formula (X), Formula (1-A), Formula (A-1), Formula (A-2), Formula (B-1), or Formula (B-2), or a pharmaceutically acceptable salt thereof, has keratolytic effects (e.g., reduces disulfide (S—S) bonds) (e.g., in any environment described herein).

In some embodiments, the dermal, ocular, and/or periocular disorders treated by administering a composition or compound provided herein are disorders that have multifactorial etiologies and/or interactions, such as bacterial or parasitic (e.g., demodex) infections or infestations. In some instances, a disorder treated according to any method provided herein is a disorder caused by a demodex infection or infestation, such as involving one or more symptom of the infestation or the infection in an individual. In some embodiments, provided herein are compounds (and compositions comprising such compounds) that have multifunctional efficacies, such as when administered to the skin and/or in or around the eye (e.g., to the ocular surface, the eyelid, such as the eyelid margin or the inner surface of the eyelid).

In some embodiments, provided herein is a method of treating a (e.g., demodex) infection, infestation, or symptoms thereof by administering (e.g., ocular, periocular, dermal) (e.g., therapeutically effective amount of) a compound or composition provided herein to an individual (e.g., in need thereof). In some instances, the infection, infestation, or symptoms thereof is, at least partially, associated with a histamine response in the individual (e.g., in need thereof). In some instances, the infection, infestation, or symptoms thereof is, at least partially, associated with keratinized material (or the buildup or blockage thereof (e.g., of the meibomian glands)) in the individual (e.g., in need thereof). In some embodiments, provided herein is a method of treating a demodex infestation, infection, or symptoms thereof, such as inflammation and/or hyperkeratosis (e.g., of the eye or skin). In some embodiments, provided herein is a method of treating a demodex infestation, infection, or symptoms thereof and/or hyperkeratosis (e.g., of the eye or skin). In some embodiments, provided herein is a method of treating inflammation and/or hyperkeratosis (e.g., of the eye or skin).

In certain embodiments, provided herein are methods of treating ocular (or dermatological) disorders associated with keratosis (e.g., lid keratosis, surface ocular keratosis, and/or gland blockage—such as in MGD, an ocular allergy (e.g., keratoconjunctivitis), dry eye disease, or the like), microbial infiltration/infection (e.g., bacterial or parasitic (e.g., demodex) infiltration/infection), improper immunomodulation (e.g., dysregulated immunoreaction(s)), and/or inflammation (such as inflammation associated keratosis or not associated with keratosis). In certain instances, disorders of the skin and/or eye (and/or surround tissue/skin) are difficult to differentially diagnose and/or have multiple etiologies. For example, in some instances, it can be difficult to distinguish between ocular disorders that involve (1) inflammation only, (2) infection only, (3) inflammation and/or infection associated with keratolytic activity, (4) inflammation and/or infection associated with both keratolytic activity (e.g., inducing keratosis) and microbial infiltration, (5) keratolytic activity, but not inflammation and/or microbial infiltration, or various other combinations. In some instances, compounds and compositions provided herein can be used in such ocular and/or dermatological indications without the need for differential diagnosis (which can be difficult, e.g., because of similar symptom scores, etc.). Further, many ocular and/or dermatological disorders involve multiple etiologies, such as improper immunomodulation, inflammation, microbial infiltration, keratolytic activity, or various combinations thereof. As a result, therapeutic agents, such as those described herein, that target multiple etiologies are beneficial in providing therapeutic efficacy, such as by targeting both an underlying condition (e.g., a dysregulated immunoreaction, keratolytic activity and/or microbial infiltration) and a symptom, such as inflammation or dry eye.

In some embodiments, provided herein are compounds, compositions, methods, and formulations for the treatment of ocular (e.g., periocular) or dermatological disorders, or symptoms thereof, such as those having multifactorial etiologies (e.g., dysregulated immunoreaction(s), inflammation, keratosis, microbial infiltration/infection, or the like).

In some embodiments, provided herein are compounds, compositions, methods, and formulations for the treatment of ocular (e.g., periocular) or dermatological infections, such as (e.g., chlamydia) or parasitic (e.g., mites (e.g., demodex), worms, protozoa (e.g., amoeba (e.g., acanthamoeba or cryptosporidiosis)), onchocerciasis, lice, scabies, nematodes, or the like) infections. In some instances, the ocular or dermatological infection is associated with an (allergy) symptom, such as itching or hypersensitivity.

In specific embodiments, ocular disorders include, by way of non-limiting example, surface disorders, such as MGD, dry eye and associated inflammatory and microbial disease, an (e.g., severe) ocular allergy (e.g., keratoconjunctivitis), and a (e.g., inflammatory and/or aqueous) dry eye disease.

Provided in some embodiments herein is a method of treating a microbial (e.g., demodex) infection or infestation, inflammation, and/or hyperkeratosis, the method comprising administering to an individual (e.g., in need thereof) any compound provided herein (e.g., of any Formula provided herein) (e.g., in a therapeutically effective amount). In specific embodiments, the microbial (e.g., demodex) infection or infestation, inflammation, and/or hyperkeratosis is a microbial (e.g., demodex) infection or infestation, inflammation, and/or hyperkeratosis of the eye, periocular structures (e.g., eyelid), and/or skin.

Provided in some embodiments herein is use of a compound having a structure represented by a formula described herein, such as Formula (X), Formula (1-A), Formula (A-1), Formula (A-2), Formula (B-1), or Formula (B-2), for treating a (e.g., keratolytic) dermal or ocular disease or disorder (e.g., the disease or disorder being associated with keratin production and/or buildup) in an individual (e.g., in need thereof).

Provided herein is a method of treating a dermal or an ocular disease or disorder in an individual, comprising administering to the individual a compound or composition described herein.

In some embodiments, the dermal or the ocular disease or disorder is associated with keratosis, microbial infiltration, microbial infection, inflammation, or any combination thereof.

In some embodiments, the dermal or the ocular disease or disorder is or is associated with an infection.

In some embodiments, the disease or disorder is or is associated with a bacterial (e.g., chlamydia) or parasitic (e.g., mites (e.g., demodex), worms, protozoa (e.g., amoeba (e.g., acanthamoeba or cryptosporidiosis)), onchocerciasis, lice, scabies, nematodes, or the like) infection.

In some embodiments, the disease or disorder is or is associated with demodex mites (e.g., Demodex Brevis, Demodex folicularum, and Demodex canis).

In some embodiments, the dermal or the ocular disease or disorder is a demodex infestation or infection.

In some embodiments, the dermal or the ocular disease or disorder is an allergy or symptom thereof, such as itching or hypersensitivity.

Provided in some embodiments herein is a method of treating a dermatological or an ophthalmic disease or disorder in an individual in need of thereof, comprising administering to the individual in need thereof a composition comprising any compound provided herein, such as a compound represented by any structure herein, such as, for example, Formula (X), Formula (1-A), Formula (A-1), Formula (A-2), Formula (B-1), or Formula (B-2), or a pharmaceutically acceptable salt thereof. In some embodiments, the dermatological or ophthalmic disease or disorder is a microbial (e.g., demodex) infection or infestation, inflammation, and/or hyperkeratosis of the eyes or skin (e.g., the ocular surface). In some embodiments, the dermatological or ophthalmic disease or disorder is selected from the group consisting of meibomian gland dysfunction (MGD), (e.g., inflammatory and/or aqueous) dry eye disease (DED), blepharitis, seborrheic blepharitis, and an ocular allergy (e.g., a severe ocular allergy). In some embodiments, the dermatological or ophthalmic disease or disorder is inflammation or hyperkeratosis (e.g., of the eyes or skin).

In some embodiments, the dermatological or ophthalmic disease or disorder is selected from the group consisting of an (ocular) allergy or a dry eye disease.

In some embodiments, the dermatological or ophthalmic disease or disorder is an ocular allergy. In some embodiments, the dermatological or ophthalmic disease or disorder is a severe ocular allergy. In some embodiments, the dermatological or ophthalmic disease or disorder is keratoconjunctivitis.

In some embodiments, the dermatological or ophthalmic disease or disorder is a dry eye disease. In some embodiments, the dermatological or ophthalmic disease or disorder is an inflammatory and/or aqueous dry eye disease. In some embodiments, the dermatological or ophthalmic disease or disorder is an inflammatory dry eye disease. In some embodiments, the dermatological or ophthalmic disease or disorder is an aqueous dry eye disease.

In some embodiments, the ophthalmic disease or disorder is selected from dry eye, lid wiper epitheliopathy (LWE), contact lens discomfort (CLD), contact lens discomfort, dry eye syndrome, evaporative dry eye syndrome, aqueous deficiency dry eye syndrome, blepharitis, keratitis, meibomian gland dysfunction, conjunctivitis, lacrimal gland disorder, inflammation of the anterior surface of the eye, infection of the anterior surface of the eye, infection of the lid, demodex lid infestation, lid wiper epitheliopathy and autoimmune disorder of the anterior surface of the eye. In some embodiments, the ophthalmic disease or disorder is a demodex lid infestation.

In some embodiments, provided herein is a method of treating an ocular (e.g., peri-ocular) or dermatological indication (e.g., associated with a (dysregulated) immunoreaction, keratolytic activity, inflammation, and/or microbial infiltration), the method comprising administering a therapeutically effective amount of a compound or composition provided herein. In some embodiments, a composition provided herein (e.g., used in a method provided herein) comprises a compound provided herein in a therapeutically effective amount (e.g., at a concentration effective to treat a (dysregulated) immunoreaction, keratosis/keratolytic activity, inflammation, and/or microbial infiltration), in the eye, surrounding tissue, or skin.

In some embodiments, provided herein are compositions and methods for the treatment of ocular and periocular conditions that have multifactorial etiologies and interactions.

### INCORPORATION BY REFERENCE

All publications, patents, and patent applications mentioned in this specification are herein incorporated by reference for the specific purpose identified herein.

### DETAILED DESCRIPTION OF THE INVENTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(Ra)S(O)ₜR^{a} (where t is 1 or 2), —S(O)OR^{a} (where t is 1 or 2), —S(O)ₜR^{a} (where t is 1 or 2) and —S(O)ₜN(R^{a})₂ (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(Ra)₂, —R^{b}—O—R^{c}—C(O)N(R^{a})₂, —R^{b}—N(Ra)C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each Ra is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R¹—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O) OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkenyl” refers to a radical of the formula —R^{c}-carbocyclyl where R^{c} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O) OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O) R^{a} (where t is 1 or 2), —R^{b}—S(O)ₜR^{a} (where t is 1 or 2), —R^{b}—S(O)ₜOR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of a optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

“Pharmaceutically acceptable salt” includes both acid and base addition salts. A pharmaceutically acceptable salt of any one of the pharmacological agents described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “keratinized obstruction,” as used herein, generally refers to a blockage of the meibomian gland, regardless of the location of the blockage. In some embodiments, the blockage is complete, whereas in other embodiments, the blockage is partial. Regardless of the degree of blockage, such keratinized obstruction leads to meibomian gland dysfunction. In some embodiments, the keratinized obstruction is composed of keratinized material and lipids. In some embodiments, the keratinized obstruction is a blockage at the meibomian gland orifice and excretory duct. In some embodiments, the keratinized obstruction is caused by keratinization of the epithelium at the lid margin and meibomian gland. In certain instances, the keratin obstruction is influenced by the migration or aberrant differentiation of stem cells. In some embodiments, the keratinized obstruction results in reduced delivery of oil to the lid margin and tear film, and stasis inside the meibomian gland that causes increased pressure, resultant dilation, acinar atrophy, and low secretion. In certain instances, keratinization of the meibomian gland causes degenerative gland dilation and atrophy.

The term, “meibomian gland dysfunction,” as used herein, refers to chronic, diffuse abnormality of the meibomian glands, that is characterized by terminal duct obstruction or qualitative or quantitative changes in the glandular secretion, or both. MGD may result in alteration of the tear film, eye irritation symptoms, inflammation, or ocular surface disease. The most prominent aspects of MGD are obstruction of the meibomian gland orifices and terminal ducts and changes in the meibomian gland secretions.

The meibomian glands are large sebaceous glands located in the eyelids, and unlike skin, are unassociated with hair. The meibomian glands produce the lipid layer of the tear film that protects it against evaporation of the aqueous phase. The meibomian gland orifice is located on the epithelial side of the lid margin, and can be a few hundred microns from the mucosal side. The glands are located on both upper and lower eyelids, with higher amounts of the glands on the upper eyelid. A single meibomian gland is composed of clusters of secretory acini that are arranged circularly around a long central duct and connected to it by short ductules. The terminal part of the central duct is lined by an ingrowth of the epidermis that covers the free lid margin and forms a short excretory duct that opens as an orifice at the posterior part of the lid margin just anterior to the mucocutaneous junction near the inner lid border. The oily secretion composed of lipids is synthesized within the secretory acini. The lipid secretion is a liquid at near body temperature and is delivered to the skin of the lid margin as a clear fluid, called “meibum.” It forms shallow reservoirs on the upper and lower lid margins, and consists of a complex mixture of cholesterol, wax, cholesteryl esters, phospholipids, with small amounts of triglycerides, triacylglycerols, and hydrocarbons. The separate meibomian glands are arranged in parallel, and in a single row throughout the length of the tarsal plates in the upper and lower lids. The extent of the glands corresponds roughly to the dimensions of the tarsal plates.

Ocular surface diseases are a group of diseases including, but not limited to, dry eye syndrome (including evaporative DES and/or aqueous deficiency DES), blepharitis, keratitis, meibomian gland dysfunction, conjunctivitis, lacrimal gland disorder, contact lens related conditions and inflammatory, infectious, or autoimmune diseases or disorders of the anterior surface of the eye.

In some instances, meibomian gland dysfunction (MGD) is a chronic, diffuse abnormality of the meibomian glands, which can be characterized by terminal duct obstruction and/or qualitative/quantitative changes in the glandular secretion. Terminal duct obstruction is caused by hyperkeratinization of the ductal epithelium (Nichols et al, Inv. Oph. & Vis. Sci. (2011); 52(4):1922-1929). These alterations in both meibum quality and expression may result in alteration of the tear film, symptoms of eye irritation, and ocular surface disease such as evaporative dry eye. The principal clinical consequence of MGD is evaporative dry eye syndrome and large population based studies (i.e., Bankok Study and the Shihpai Eye Study) estimate that over 60% of patients with dry eye symptoms also have MGD (Schaumberg et al, Investigative Ophthalmology and Visual Science. (2011); 52(4):1994-2005).

MGD is a leading contributor of dry eye syndrome. The occurrence of dry eye syndrome is widespread and affects about 20 million patients in the United States alone. Dry eye syndrome is a disorder of the ocular surface resulting from either inadequate tear production or excessive evaporation of moisture from the surface of the eye. Tears are important to corneal health because the cornea does not contain blood vessels, and relies on tears to supply oxygen and nutrients. Tears and the tear film are composed of lipids, water, and mucus, and disruption of any of these can cause dry eye. An inadequate amount of lipids flowing from the meibomian glands as caused by a keratinized obstruction, may cause excessive evaporation, thereby causing dry eye syndrome.

Currently there are no approved pharmacological agents useful for the treatment of MGD. The recognition that terminal duct obstruction from hyperkeratinization of the ductal epithelium on meibomian glands is a core mechanism behind meibomian gland dysfunction (MGD) is consistent with clinical experience demonstrating that effective treatments for MGD require resolution of ductal obstruction and evacuation of glandular contents (Nichols et al, 2011; Lane et al, 2012; Blackie et al, 2015). Warm compresses and thermal/mechanical devises (e.g., LipiFlow) are used in an attempt to raise the internal temperature of the meibomian glands over the normal melting point for meibum (i.e., 32° C. to 40° C.) in an attempt to resolve terminal duct obstruction (Lane et al, 2012). Unfortunately, warm compresses are unable to achieve this benefit for severely obstructed glands which can having a melting point >40° C. Current technology for removing keratinized obstruction of the meibomian gland also includes physical removal methods (e.g., debridement and gland probing), which are quite painful to patients.

Subsequent to a period of MGD, various stages of immune, inflammatory, and/or microbial (e.g., demodex) disease at the ocular surface are frequently observed because meibomian gland obstruction can cause a cascade of events that include further deterioration of the glands (Knop, IOVS, 2011) from stasis of the meibum in the secretory glands, mechanical pressure and stress from glandular obstruction, and increased bacterial growth that is associated with the downstream release of bacterial lipases, toxic mediators, and/or inflammatory mediators. All these factors reduce the quality and/or quantity of meibum the glands can release which in turn can cause chronic mechanical traumatization of the conjunctival, corneal and eyelid tissues which will lead to further tissue damage and the release of inflammatory mediators. Thus, many patients suffering from MGD also have inflammatory disease affecting their conjunctiva, cornea, larcrimal gland, lids or goblet cells causing comorbid conditions such as dry eye syndrome or blepharitis for which there is an unmet medical need.

For example, literature has used the terms posterior blepharitis and MGD as if they were synonymous, but these terms are not interchangeable. Posterior blepharitis describes inflammatory conditions of the posterior lid margin, of which MGD can be one possible cause. In its earliest stages, MGD may not be associated with clinical signs characteristic of posterior blepharitis. At this stage, affected individuals may be symptomatic, but alternatively, they may be asymptomatic and the condition regarded as subclinical. As MGD progresses, symptoms develop and lid margin signs, such as changes in meibum expressibility and quality and lid margin redness, may become more visible. At this point, an MGD-related posterior blepharitis is said to be present.

In some instances, altered meibomian gland secretion is detected by physically expressing the meibomian glands by applying digital pressure to the tarsal plates. In subjects without MGD, the meibum is a pool of clear oil. In MGD, both the quality and expressibility of the expressed material is altered. The altered meibum is also known as meibomian excreta and is made up of a mixture of altered secretions and keratinized epithelial material. In MGD, the quality of expressed lipid varies in appearance from a clear fluid, to a viscous fluid containing particulate matter and densely opaque, toothpaste-like material. The meibomian orifices may exhibit elevations above surface level of the lid, which is referred to as plugging or pouting, and is due to obstruction of the terminal ducts and extrusion of a mixture of meibomian lipid and keratinized material.

In some instances, obstructive MGD is characterized by all or some of the following: 1) chronic ocular discomfort, 2) anatomic abnormalities around the meibomian gland orifice (which is one or more of the following: vascular engorgement, anterior or posterior displacement of the mucocutaneous junction, irregularity of the lid margin) and 3) obstruction of the meibomian glands (obstructive findings of the gland orifices by slit lamp biomicroscopy (pouting, plugging or ridge), decreased meibum expression by moderate digital pressure).

Methods for assessing and monitoring MGD symptoms may include, but are not limited to patient questionnaires, meibomian gland expression, tear stability break up time, and determining the number of patent glands as seen by digital expression.

In some embodiments, the symptoms of a patient are assessed by asking the patient a series of questions. Questionnaires allow the assessment of a range of symptoms associated with ocular discomfort. In some embodiments, the questionnaire is the SPEED questionnaire. The SPEED questionnaire assesses frequency and severity of a patient's dry eye symptoms. It examines the occurrence of symptoms on the current day, past 72 hours and past three months. A SPEED score is tallied based on the patient's answers to the questions, to give a range of severity of the patient's symptoms. The SPEED questionnaire includes questions such as the following: 1) what dry eye symptoms are you experiencing, and when do they occur? 2) how frequently do you experience dryness, grittiness, or scratchiness in your eyes? 3) how often do you experience soreness or irritation of the eyes? 4) how often do you experience burning or watering of the eyes? 5) how often do you experience eye fatigue? and 6) how severe are the symptoms?

Meibomian gland expressibility is optionally determined to assess the meibomian gland function. In normal patients, meibum is a clear to light yellow oil. Meibum is excreted from the glands when digital pressure is placed on the glands. Changes in meibomian gland expressibility are one potential indicator of MGD. In some embodiments, during expression, quantifying the amount of physical force applied during expression is monitored in addition to assessing lipid volume and lipid quantity.

Tear stability break up time (TBUT) is a surrogate marker for tear stability. Tear film instability is a core mechanism in dry eye and MGD. Low TBUT implies a possibility of lipid layer compromise and MGD. TBUT is optionally measured by examining fluorescein breakup time, as defined as the time to initial breakup of the tear film after a blink. Fluorescein is optionally applied by wetting a commercially available fluorescein-impregnated strip with saline, and applied to the inferior fornix or bulbar conjuctiva. The patient is then asked to blink several times and move the eyes. The break up is then analyzed with a slit lamp, a cobalt blue filter, and a beam width of 4 mm. The patient is instructed to blink, and the time from upstroke of the last blink to the first tear film break or dry spot formation is recorded as a measurement.

Other methods for assessing MGD symptoms, include but are not limited to, Schirmer test, ocular surface staining, lid morphology analysis, meibography, meibometry, interferometry, evaporimetry, tear lipid composition analysis, fluorophotometry, meiscometry, osmolarity analysis, indices of tear film dynamics, evaporation and tear turnover.

Treatments for MGD can include lid warming, lid massage, lid hygiene, lid expression and meibomian gland probing. Pharmacological methods, prior to those described herein, have not been used.

Lid hygiene is considered the primary treatment for MGD and consists of three components: 1) application of heat, 2) mechanical massage of eyelids and 3) cleansing the eyelid. Eyelid warming procedures improve meibomian gland secretion by melting the pathologically altered meibomian lipids. Warming is achieved by warm compresses or devices. Mechanical lid hygiene includes the use of scrubs, mechanical expression and cleansing with various solutions of the eyelashes and lid margins. Lid margins are optionally also cleansed with hypoallergenic bar soap, dilute infant shampoo or commercial lid scrubs. Physical expression of meibomian glands is performed in a physician's office or is performed by the patient at home. The technique varies from gentle massage of the lids against the eyeball to forceful squeezing of the lids either against each other or between a rigid object on the inner lid surface and a finger, thumb, or rigid object (such as a glass rod, cotton swab, or metal paddle) on the outer lid surface. The rigid object on the inner lid surface protects the eyeball from forces transferred through the eyelid during expression and to offer a stable resistance, to increase the amount of force that is applied to the glands.

Eyelid warming is limited because the warming melts the lipids, but does not address movement of the keratinized material. Further, eyelid warming induces transient visual degradation due to corneal distortion. Mechanical lid hygiene is also limited because the force needed to remove an obstruction can be significant, resulting in significant pain to the patient. The effectiveness of mechanical lid hygiene is limited by the patient's ability to tolerate the associated pain during the procedure. Other treatments for MGD are limited.

Physical opening of meibomian glands obstruction by meibomian gland expression is an acceptable method to improve meibomian gland secretion and dry eye symptoms. In addition, probing of the meibomian gland canal has been used to open the obstructed canal. Both methods, expression and probing, are limited, however, by the pain induced by the procedure, the possible physical insult to the gland and canal structures and their short lived effect estimated at days and weeks. Therefore, methods are needed to improve patient comfort, which will not cause harm to the meibomian glands and canals, that will reduce the dependency on frequent office visits and improve secretion of meibum.

U.S. Pat. No. 9,463,201 entitled, “Compositions and methods for the treatment of meibomian gland dysfunction” describes a method for treating meibomian gland dysfunction involving the topical administration of a therapeutically-effective amount of at least one keratolytic agent in an ophthalmically-acceptable carrier. The patent includes keratolytic agents that are inorganic selenium (Se) compounds such as selenium disulfide (SeS₂) or organoselenium compounds such as Ebselen (2-Phenyl-1,2-benzoselenazol-3-one). This agent would treat the underlying cause of MGD, but not a “plus” inflammatory disease (or demodex infestation) as described by the DEWS report on MGD.

The role of inflammation in the etiology of MGD is controversial. The terms posterior blepharitis and MGD are not synonymous. Posterior blepharitis describes inflammatory conditions of the posterior lid margin and has various causes, of which MGD can be one possible cause (Nichols et al 2011). In its earliest stages, MGD is not associated with clinical signs characteristic of posterior blepharitis. As MGD progresses, an MGD-related posterior blepharitis is said to be present. MGD-related posterior blepharitis affects the meibomian glands and meibomian gland orifices. MGD-related posterior blepharitis is characterized by flora changes, esterase and lipase release, lipid changes, and eyelid inflammation. Hyperkeratinization of the meibomian gland epithelium (thickening of the lining of the glands) may lead to obstruction and a decrease in the quantity of meibomian gland secretions and may be responsible for MGD-related posterior blepharitis. Diagnosis of MGD-related posterior blepharitis includes meibomian gland expression with demonstration of an altered quality of expressed secretions, and/or by a loss of gland functionality (decreased or absent expressibility). The TFOS report on Meibomian Gland Disease specifically notes that anterior blepharitis and exacerbated inflammatory ocular surface disease are “plus” diseases to MGD which are managed by topical, ocular steroids (Nichols et al 2011). Since these “plus” conditions can be present in various levels of severity from early to late MGD there is a need for treatments and/or combinations of treatments that can target both the underlying non-inflammatory pathophysiology of MGD and inflammation associated with these comorbid conditions.

MGD-related inflammatory eye disease may comprise a different mechanism than blepharitis-related MGD. MGD-related inflammatory eye disease is characterized by an inflammatory cascade involving activation and migration of T lymphocytes to the inflamed tissue. T lymphocyte infiltration may result in lacrimal gland stimulation and upregulation of cytokines. Exemplary cytokines that may be involved in MGD-related inflammatory eye disease include, but are not limited to, interleukin-1, interleukin-4, interleukin-6, inteleukin-8, interferon gamma, macrophage inflammatory protein 1 alpha, and tumor necrosis factor alpha. Kinase pathways including the mitogen activated protein kinase (MAPK) pathway are also activated in the inflammatory cascade. The inflammatory process results in loss of mucin-producing goblet cells and destruction of the ocular surface that can lead to further damage.

Dry eye syndrome, also known as keratoconjunctivitis sicca (KCS), is considered a self-sustaining disease that is progressively disconnected from its initial cause. Dry eye syndrome is associated with inflammation at the ocular surface and periocular tissue. Inflammation is characterized by the activation and migration of T lymphocytes to the inflamed tissue including in the conjunctiva and lacrimal glands. Inflammatory cytokines, chemokines, and matrix metalloproteinase have also been identified as being increased.

Animal models of dry eye disease have been established and reviewed (Barabino, et al, (Invest. Ophthalmol. Vis. Sci. 2004, 45:1641-1646)). Barabino, et al, (Invest. Ophthalmol. Vis. Sci. 2005, 46:2766-2771) described a model wherein exposure of normal mice to a low-humidity environment in a controlled-environment chamber leads to significant alterations in tear secretion, goblet cell density, and acquisition of dry eye-related ocular surface signs. However, no single animal model adequately accounts for the immune, endocrine, neuronal and environmental factors which contribute to dry eye pathogenesis.

Anti-inflammatory agents may be used to treat ocular surface diseases or disorders including dry eye syndrome. In some instances, corticosteroids are an effective anti-inflammatory therapy in dry eye disease. For example, in a 4-week, double-masked, randomized study in 64 patients with dry eye and delayed tear clearance, loteprednol etabonate 0.5% ophthalmic suspension (Lotemax [Bausch and Lomb, Rochester, NY]), QID, was found to be more effective than its vehicle in improving some signs and symptoms (Pflugfelder et al, Am J Ophthalmol (2004); 138:444-57). The TFOS 2007 report on dry eye disease went so far as to conclude that, “In the US Federal Regulations, ocular corticosteroids receiving “class labeling” are indicated for the treatment “ . . . of steroid responsive inflammatory conditions of the palpebral and bulbar conjunctiva, cornea and anterior segment of the globe such as allergic conjunctivitis, acne rosacea, superficial punctate keratitis, herpes zoster keratitis, iritis, cyclitis, selected infective conjunctivitis, when the inherent hazard of steroid use is accepted to obtain an advisable diminution in edema and inflammation.” KCS, in some instances, is included in this list of steroid-responsive inflammatory conditions (Therapy Subcommittee of the International Dry Eye WorkShop, 2007. Management and Therapy of Dry Eye Disease: Report of the Management and Therapy Subcommittee of the International Dry Eye WorkShop (2007). 2007; 5: 163-178).” While the US FDA does not agree with this conclusion, short courses of steroids, especially Lotemax, can be commonly used to treat inflammation associated with dry eye disease.

Other anti-inflammatory agents include nonsteroidal anti-inflammatory drugs (NSAIDs). NSAIDs inhibit the activity of cyclooxygenases including cyclooxygenase-1 (COX-1) and cyclooxygenase-2 (COX-2), which are enzymes involved in the synthesis of prostaglandins and thromboxanes from arachidonic acid. Prostaglandin and thromboxane signaling are involved in inflammation and immune modulation. In some cases, NSAIDs are used for treating dry eye disease by treating the inflammation at the ocular surface.

In some instances, provided herein is a compound that comprises a radical of an antiparasitic agent described herein (e.g., moxidectin or ivermectin). In some instances, provided herein is a compound that comprises a radical of an antiparasitic agent described herein (e.g., moxidectin or ivermectin) and a radical of a keratolytic agent described herein. In some instances, provided herein is a compound that comprises a radical of an antiparasitic agent described herein (e.g., moxidectin or ivermectin) and a radical of a H1 antagonist described herein. In some embodiments, provided herein is a compound having a structure represented by Formula (X): Y-L-Z, wherein Y is an antiparasitic agent described herein, L is a linker described herein, and Z is a radical of a keratolytic agent described herein or a radical of a H1 antagonist described herein.

In some instances, provided herein is a compound that delivers a therapeutically effective amount of (e.g., a free form of) an anti-parasitic agent, such as an anti-parasitic agent described herein (e.g., where the anti-parasitic agent reduces the amount of a demodex mites and/or reduces or eliminates the infestation thereof) and/or (e.g., a free form of) an H1 antagonist, such as an H1 antagonist described herein. In some instances, the free form of the antiparasitic agent is ivermectin. In some instances, the free form of the antiparasitic agent is moxidectin. In some instances, the free form of the H1 antagonist is selected from the group consisting of olopatadine, cetirizine, acrivastine, bilastine, fexofenadine, levocabastine, hydroxyzine, periciazine, and quetiapine.

The chemical name for olopatadine is {(11Z)-11-[3-(dimethylamino)propylidene]-6,11-dihydrodibenzo[b,e]oxepin-2-yl}acetic acid having a molecular formula of C₂₁H₂₃NO₃ and a molecular weight of 337.419 g/mol. The structural formula of olopatadine is:

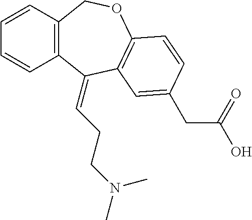 [SMILES: CN(C)CC/C=C1/c2ccccc2COc2ccc(CC(=O)O)cc21]

Olopatadine is a selective antagonist of the histamine H₁ receptor that is used to decrease the symptoms of allergic conjunctivitis (in the form of an eye drop) and allergic rhinitis (in the form of a nasal spray). The selective H1 antagonist is a mast cell stabilizer that stabilizes basophils and conjunctival mast cells and inhibits the immunologically-stimulated release of histamine, thereby inhibiting the release of inflammatory mediators, such as tryptase, prostaglandin D2, TNF-alpha, as well as pro-inflammatory cytokines. Olopatadine is a topical second-generation antihistamine containing both a tricyclic ring and an alkylamine as well as a carboxylic acid that is suitable for conjugation via condensation reactions.

The chemical name for cetirizine is (±)-[2-[4-[(4-Chlorophenyl)phenylmethyl]-1-piperazinyl]ethoxy]acetic acid having a molecular formula of C₂₁H₂₅ClN₂O₃ and a molecular weight of 388.89 g/mol. The structural formula of cetirizine is:

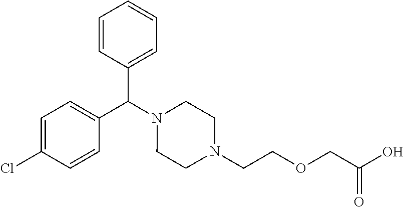 [SMILES: O=C(O)COCCN1CCN(C(c2ccccc2)c2ccc(Cl)cc2)CC1]

Cetirizine is a selective antagonist of the histamine H₁ receptor that is used to treat allergic rhinitis, dermatitis, and urticaria (in an oral form). By blocking the H₁ receptor, cetirizine relieves itching and redness which can be caused by histamine acting on the H₁ receptor. Cetirizine is a second-generation antihistamine that contains a piperazine and two aromatic (e.g., phenyl) rings connected to a central carbon as well as a carboxylic acid that is suitable for conjugation via condensation reactions.

The chemical name for acrivastine is (E)-3-{6-[(E)-1-(4-methylphenyl)-3-pyrrolidin-1-yl-prop-1-enyl]pyridin-2-yl}prop-2-enoic acid having a molecular formula of C₂₂H₂₄N₂O₂ and a molecular weight of 348.446 g/mol. The structural formula of acrivastine is:

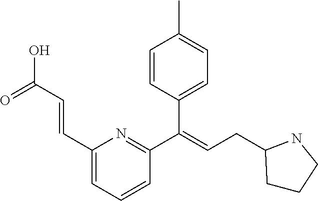 [SMILES: Cc1ccc(/C(=C\CC2CCCN2)c2cccc(/C=C/C(=O)O)n2)cc1]

Acrivastine is a selective antagonist of the histamine H₁ receptor that blocks the effects of histamine and reduces symptoms such as itching, watery eyes, a running or blocked nose, sneezing and skin rashes. Acrivastine is a second-generation antihistamine that contains an alkylamine and two aromatic rings connected to a central carbon as well as a carboxylic acid that is suitable for conjugation via condensation reactions.

The chemical name for bilastine is 2-[4-(2-{4-[1-(2-Ethoxyethyl)-1H-benzimidazol-2-yl]-1-piperidinyl}ethyl)phenyl]-2-methylpropanoic acid having a molecular formula of C₂₈H₃₇N₃O₃ and a molecular weight of 463.622 g/mol. The structural formula of bilastine is:

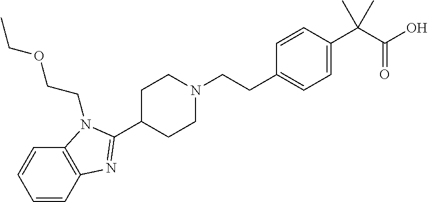 [SMILES: CCOCCn1c(C2CCN(CCc3ccc(C(C)(C)C(=O)O)cc3)CC2)nc2ccccc21]

Bilastine is a selective antagonist of the histamine H₁ receptor that is used to treat uricaria and allergic conjunctivitis (in an oral form) as well as reduce symptoms associated therewith, such as itching, watery eyes, and the like. Bilastine is a second-generation antihistamine that contains an alkylamine and an ethanolamine as well as a carboxylic acid that is suitable for conjugation via condensation reactions.

The chemical name for fexofenadine is (±)-4-[1-Hydroxy-4-[4-(hydroxydiphenylmethyl)-1-piperidinyl]-butyl]-α, α-dimethyl benzeneacetic acid having a molecular formula of C₃₂H₃₉NO₄ and a molecular weight of 501.667 g/mol. The structural formula of fexofenadine is:

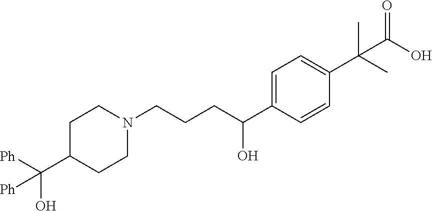 [SMILES: CC(C)(C(=O)O)c1ccc(C(O)CCCN2CCC(C(O)(c3ccccc3)c3ccccc3)CC2)cc1]

Fexofenadine is a selective antagonist of the histamine H₁ receptor that is used to treat allergy symptoms, such as uricaria and allergic conjunctivitis (in an oral form), and thereby providing relief from runny nose, itchy eyes or skin, and sneezing. Fexofenadine is a second-generation antihistamine that contains an alkylamine as well as a carboxylic acid that is suitable for conjugation via condensation reactions.

The chemical name for levocabastine is (3S,4R)-1-[cis-4-cyano-4-(4-fluorophenyl)cyclohexyl]-3-methyl-4-phenyl-4-piperidinecarboxylic acid having a molecular formula of C₂₆H₂₉FN₂O₂ and a molecular weight of 420.528 g/mol. The structural formula of levocabastine is:

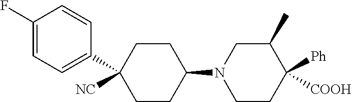 [SMILES: C[C@@H]1CN([C@H]2CC[C@](C#N)(c3ccc(F)cc3)CC2)CC[C@]1(C(=O)O)c1ccccc1]

Levocabastine is a selective H1 antagonist that is used in the form of eye drops for treating allergic conjunctivitis and other similar allergic ocular conditions. Levocabastine is a second-generation antihistamine that contains an alkylamine as well as a carboxylic acid that is suitable for conjugation via condensation reactions.

The chemical name for hydroxyzine is (±)-2-(2-{4-[(4-chlorophenyl)-phenylmethyl]piperazin-1-yl}ethoxy)ethanol having a molecular formula of C₂₁H₂₇C₁N₂O₂ and a molecular weight of 374.91 g/mol. The structural formula of hydroxyzine is:

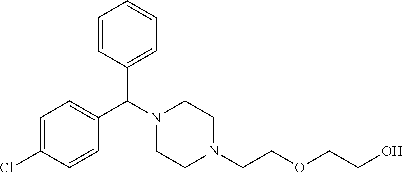 [SMILES: OCCOCCN1CCN(C(c2ccccc2)c2ccc(Cl)cc2)CC1]

Hydroxyzine is a selective antagonist of the histamine H₁ receptor that is used to treat itchiness, chronic urticaria, atopic or contact dermatoses, and histamine-mediated pruritus. Hydroxyzine is an antihistamine that contains a piperazine and two aromatic (e.g., phenyl) rings connected to a central carbon as well as a hydroxyl that is suitable for conjugation, such as via condensation reactions.

Described herein are compounds (e.g., keratolytic conjugates and/or dual acting-agents) which address simultaneously the non-inflammatory keratosis (e.g., keratolytic blockage) component of dermal and/or ocular diseases or disorders described herein (e.g., MGD) and the improper modulation of histamine and/or inflammation associated with an infection, such as a bacterial or parasitic (e.g., demodex) infection or infestation. In some embodiments, a compound provided herein is useful as either an acute therapy (e.g., by a trained specialist or physician) or as a chronic therapy (e.g., in the hands of a patient, or alternatively, by a trained specialist or physician). A compound provided herein is tested, in some embodiments, using the assays and methods described herein (e.g., as described in the examples). In some embodiments, a compound provided herein represents a significant advance as the first-order metabolites obtained from metabolism of the agents are operative against both the keratolytic and the antihistamine and/or inflammatory component of diseases, such as, for example, ocular allergies, dry eye disease, and the like.

Provided in some embodiments herein is a compound having a structure represented by Formula (X):

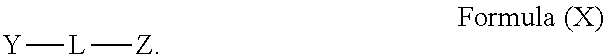

In some embodiments, Y is a radical of an anti-parasitic agent described herein. In some embodiments, Z is a radical of a keratolytic agent described herein or a radical of an H1 antagonist described herein. In some embodiments, Z is a radical of a keratolytic agent described herein. In some embodiments, Z is a radical of an H1 antagonist described herein. In some embodiments, L is a linker described herein.

In some embodiments, Y has a structure represented by Formula (1-A):

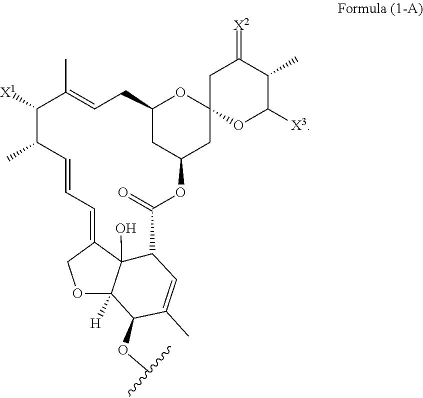

In some embodiments, X¹ is absent.

In some embodiments, X¹ is:

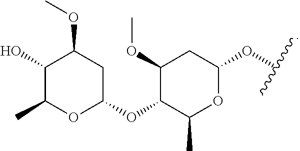

In some embodiments, X² is absent or ═NOCH₃.

In some embodiments, X² is absent.

In some embodiments, X² is ═NOCH₃.

In some embodiments, X³ is substituted or unsubstituted alkyl. In some embodiments, X³ is substituted alkyl. In some embodiments, X³ is unsubstituted alkyl. In some embodiments, X³ is unsubstituted alkyl.

In some embodiments, X³ is branched alkyl.

In some embodiments, X³ is substituted or unsubstituted branched alkyl. In some embodiments, X³ is substituted branched alkyl. In some embodiments, X³ is unsubstituted branched alkyl. In some embodiments, X³ is —C(CH₃)═CHCH(CH₃)₂.

In some embodiments, Y is a radical of moxidectin.

Unless stated otherwise, moxidectin has the following structure:

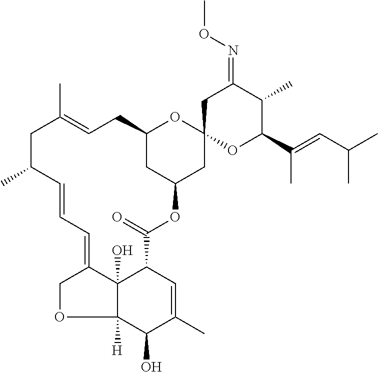 [SMILES: CO/N=C1\C[C@]2(C[C@@H]3C[C@@H](C/C=C(\C)C[C@@H](C)/C=C/C=C4\CO[C@@H]5[C@H](O)C(C)=C[C@@H](C(=O)O3)[C@]45O)O2)O[C@H](/C(C)=C/C(C)C)[C@H]1C]

In some embodiments, Y is not a radical of ivermectin.

In some embodiments, Y is not a radical of metronidazole.

In some embodiments, Y does not have the following structure:

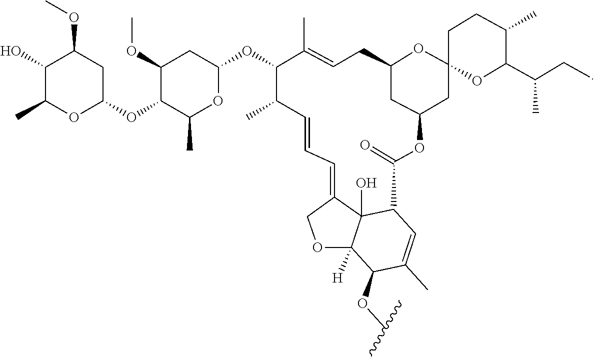

In some embodiments, does not have the following structure:

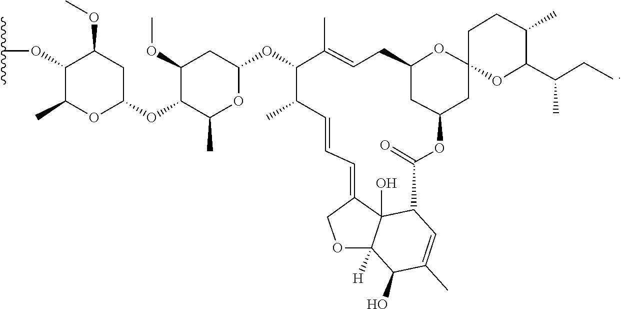

In some embodiments, Z is a radical of a keratolytic agent described herein.

In some embodiments, Z is a radical of an H1 antagonist described herein.

In some embodiments, L is any linker described herein.

Provided in some embodiments herein is a compound having a structure represented by Formula (A-1):

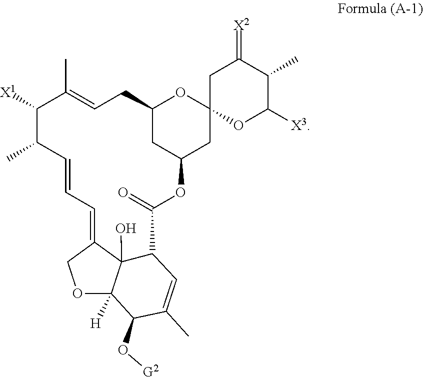

In some embodiments, X¹ is:

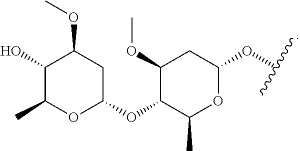

In some embodiments, X¹ is absent. In some embodiments, X² is absent or ═NOCH₃. In some embodiments, X² is absent. In some embodiments, X² is ═NOCH₃. In some embodiments, X³ is substituted or unsubstituted alkyl. In some embodiments, X³ is substituted alkyl. In some embodiments, X³ is unsubstituted alkyl. In some embodiments, X³ is unsubstituted alkyl. In some embodiments, X³ is branched alkyl. In some embodiments, X³ is substituted or unsubstituted branched alkyl. In some embodiments, X³ is substituted branched alkyl. In some embodiments, X³ is unsubstituted branched alkyl. In some embodiments, X³ is —C(CH₃)═CHCH(CH₃)₂. In some embodiments, G¹ is described elsewhere herein. In some embodiments, G¹ is hydrogen, substituted or unsubstituted alkyl, or -L¹-D¹. n some embodiments, G² is described elsewhere herein. In some embodiments, G² is hydrogen, substituted or unsubstituted alkyl, or -L²-D². In some embodiments, at least one of G¹ or G² is -L¹-D¹ or -L² D². In some embodiments, D¹ and D² are described elsewhere herein. In some embodiments, D¹ is a radical of an H1 antagonist. In some embodiments, D² is a radical of an H1 antagonist. In some embodiments, L¹ and L² are described elsewhere herein. In some embodiments, L¹ is a linker described herein. In some embodiments, L² is a linker described herein.

In some embodiments, X¹ is absent.

In some embodiments, X² is absent or ═NOCH₃.

In some embodiments, X² is absent.

In some embodiments, X² is ═NOCH₃.

In some embodiments, X³ is substituted or unsubstituted alkyl. In some embodiments, X³ is substituted alkyl. In some embodiments, X³ is unsubstituted alkyl. In some embodiments, X³ is unsubstituted alkyl.

In some embodiments, X³ is branched alkyl.

In some embodiments, X³ is substituted or unsubstituted branched alkyl. In some embodiments, X³ is substituted branched alkyl. In some embodiments, X³ is unsubstituted branched alkyl. In some embodiments, X³ is —C(CH₃)═CHCH(CH₃)₂.

In some embodiments, G² is -L²-D², L² and D² being described elsewhere herein.

In some embodiments, the compound does not have a structure represented by Formula (I), which is described elsewhere herein.

Provided in some embodiments herein is a compound having a structure represented by Formula (A-2):

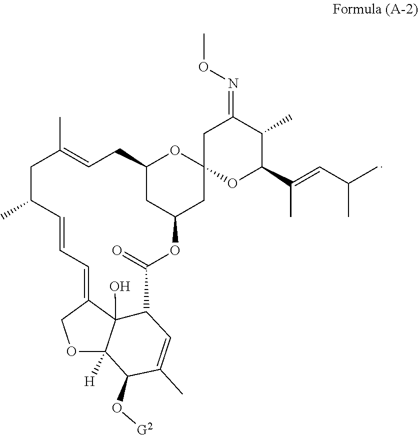

In some embodiments, G² is -L²-D². In some embodiments, D² is a radical of an H1 antagonist, such as an H1 antagonist described herein. In some embodiments, the H1 antagonist is a first-generation H1 antagonist described herein. In some embodiments, the H1 antagonist is a second-generation H1 antagonist described herein. In some embodiments, the H1 antagonist is selected from the group consisting of olopatadine, cetirizine, acrivastine, bilastine, fexofenadine, levocabastine, hydroxyzine, periciazine, and quetiapine. In some embodiments, L² is a linker, such as a linker described herein.

Provided in some embodiments herein is a compound having a structure represented by Formula (B-1):

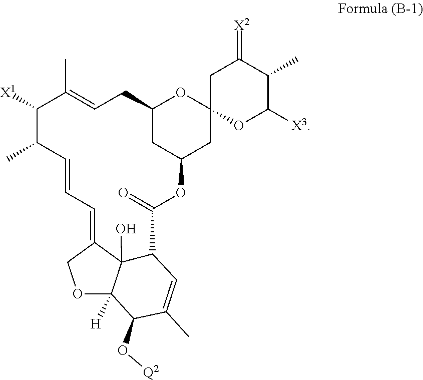

In some embodiments, X¹ is:

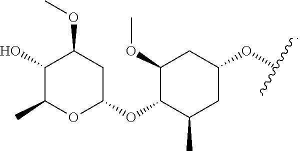

In some embodiments, X¹ is absent. In some embodiments, X² is absent or ═NOCH₃. In some embodiments, X² is absent. In some embodiments, X² is ═NOCH₃. In some embodiments, X³ is substituted or unsubstituted alkyl. In some embodiments, X³ is substituted alkyl. In some embodiments, X³ is unsubstituted alkyl. In some embodiments, X³ is unsubstituted alkyl. In some embodiments, X³ is branched alkyl. In some embodiments, X³ is substituted or unsubstituted branched alkyl. In some embodiments, X³ is substituted branched alkyl. In some embodiments, X³ is unsubstituted branched alkyl. In some embodiments, X³ is —C(CH₃)═CHCH(CH₃)₂. In some embodiments, Q¹ is described elsewhere herein. In some embodiments, Q¹ is hydrogen, substituted or unsubstituted alkyl, or -L^{A}-R¹. In some embodiments, Q² is described elsewhere herein. In some embodiments, Q² is hydrogen, substituted or unsubstituted alkyl, or -L^{B}-R². In some embodiments, at least one of Q¹ or Q² is -L^{A}-R¹ or -L^{B}-R². In some embodiments, R¹ and R² are described elsewhere herein. In some embodiments, R¹ is a radical of a keratolytic agent described herein. In some embodiments, R² is a radical of a keratolytic agent described herein. In some embodiments, L^{A} and L^{B} are described elsewhere herein. In some embodiments, L^{A} is a linker described herein. In some embodiments, L^{B} is a linker described herein.

In some embodiments, X¹ is absent.

In some embodiments, X² is absent or ═NOCH₃.

In some embodiments, X² is absent.

In some embodiments, X² is ═NOCH₃.

In some embodiments, X³ is substituted or unsubstituted alkyl. In some embodiments, X³ is substituted alkyl. In some embodiments, X³ is unsubstituted alkyl. In some embodiments, X³ is unsubstituted alkyl.

In some embodiments, X³ is branched alkyl.

In some embodiments, X³ is substituted or unsubstituted branched alkyl. In some embodiments, X³ is substituted branched alkyl. In some embodiments, X³ is unsubstituted branched alkyl. In some embodiments, X³ is —C(CH₃)═CHCH(CH₃)₂.

In some embodiments, Q² is -L^{B}-R², L^{B} and R² being described elsewhere herein.

In some embodiments, the compound does not have a structure represented by Formula (II), which is described elsewhere herein.

Provided in some embodiments herein is a compound having a structure represented by Formula (B-2):

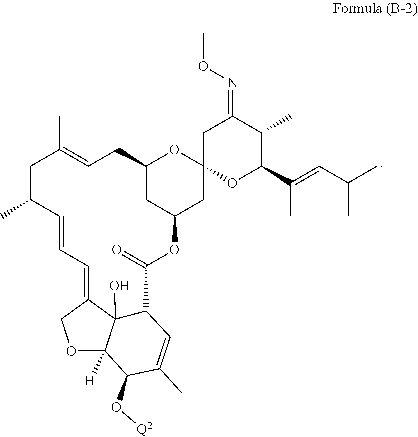

In some embodiments, Q² is -L^{B}-R². In some embodiments, R² is a radical of a keratolytic agent, such as a keratolytic agent described herein. In some embodiments, L^{B} is a linker, such as a linker described herein.

Provided in some embodiments herein is a compound having a structure represented by Formula (I):

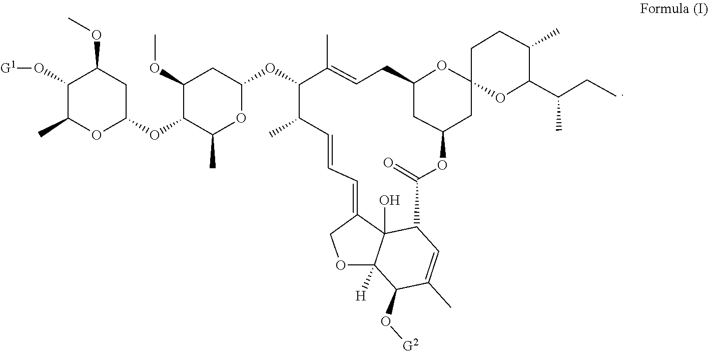

In some embodiments, G¹ is hydrogen, substituted or unsubstituted alkyl, or -L¹-D¹. In some embodiments, G² is hydrogen, substituted or unsubstituted alkyl, or -L²-D². In some embodiments, at least one of G¹ or G² is -L¹-D¹ or -L² D². In some embodiments, D¹ and D² are each independently a radical of an H1 antagonist. In some embodiments, L¹ and L² are each independently a linker. In some embodiments, the compound is a pharmaceutically acceptable salt or solvate.

In some embodiments, G¹ is hydrogen. In some embodiments, G¹ is substituted or unsubstituted alkyl. In some embodiments, G¹ is -L¹-D¹.

In some embodiments, G² is hydrogen. In some embodiments, G² is substituted or unsubstituted alkyl. In some embodiments, G² is -L² D².

In some embodiments, G¹ is -L¹-D¹ and G² is hydrogen.

In some embodiments, G¹ is hydrogen and G² is -L²-D².

In some embodiments, the H1 antagonist (G¹ or G²) is a first-generation H1 antagonist.

In some embodiments, the H1 antagonist (G¹ or G²) is a second-generation H1 antagonist.

In some embodiments, the H1 antagonist (G¹ or G²) is an ethylenediamine (H1 antagonist), ethanolamine (H1 antagonist), alkylamine (H1 antagonist), piperazine (H1 antagonist), tricyclic (H1 antagonist), or tetracyclic (H1 antagonist). In some embodiments, the H1 antagonist (G¹ or G²) is a piperazine (H1 antagonist) or a tricyclic (H1 antagonist).

In some embodiments, the H1 antagonist (G¹ or G²) comprises two aromatic rings. In some embodiments, the two aromatic rings are connected to a carbon atom, a nitrogen atom, or CO. In some embodiments, the two aromatic rings are connected to a central carbon atom, a nitrogen atom, or CO. In some embodiments, at least one of the aromatic rings is phenyl. In some embodiments, both of the aromatic rings are phenyl.

In some embodiments, the H1 antagonist (G¹ or G²) comprises a tricyclic ring.

In some embodiments, the H1 antagonist (G¹ or G²) comprises an amine. In some embodiments, the amine is substituted with alkyl. In some embodiments, the amine is substituted with methyl.

In some embodiments, the H1 antagonist (G¹ or G²) comprises a spacer. In some embodiments, the spacer is positioned between the amine and the central carbon atom, nitrogen atom, or CO. In some embodiments, the spacer is substituted or unsubstituted alkyl. In some embodiments, the spacer is linear or branched alkyl. In some embodiments, the spacer is cyclic or acyclic alkyl. In some embodiments, the spacer is saturated or unsaturated alkyl.

In some embodiments, the H1 antagonist is described herein, such as hereinabove. In some embodiments, the H1 antagonist is selected from the group consisting of olopatadine, cetirizine, acrivastine, bilastine, fexofenadine, levocabastine, hydroxyzine, periciazine, and quetiapine. In some embodiments, the H1 antagonist is selected from the group consisting of olopatadine, cetirizine, acrivastine, bilastine, fexofenadine, and levocabastine. In some embodiments, the H1 antagonist is olopatadine or cetirizine. In some embodiments, the H1 antagonist is selected from the group consisting of hydroxyzine, periciazine, and quetiapine.

Provided in some embodiments herein is a compound having a structure represented by Formula (II):

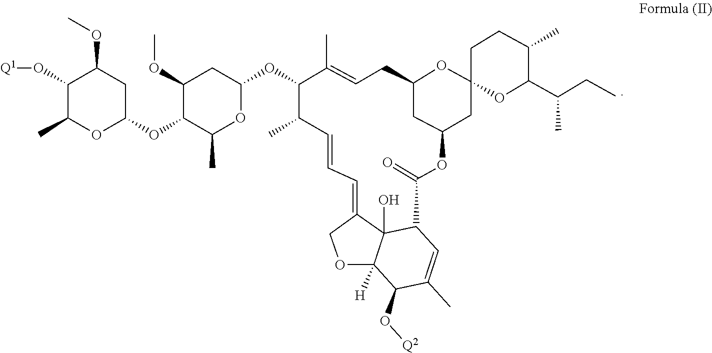

In some embodiments, Q¹ is hydrogen, substituted or unsubstituted alkyl, or -L^{A}-RI. In some embodiments, Q² is hydrogen, substituted or unsubstituted alkyl, or -L^{B}-R². In some embodiments, at least one of Q¹ or Q² is -L^{A}-R¹ or -L^{B}-R². In some embodiments, R¹ and R² are each independently a radical of a keratolytic agent. In some embodiments, L^{A} and L^{B} are each independently a linker. In some embodiments, the compound is a pharmaceutically acceptable salt or solvate.

In some embodiments, Q¹ is hydrogen. In some embodiments, Q¹ is substituted or unsubstituted alkyl. In some embodiments, Q¹ is -L^{A}-R^{I}.

In some embodiments, Q² is hydrogen. In some embodiments, Q² is substituted or unsubstituted alkyl. In some embodiments, Q² is -L^{B}-R².

In some embodiments, Q¹ is -L^{A}-R¹ and G² is hydrogen.

In some embodiments, Q¹ is hydrogen and Q² is -L^{B}R².

In some embodiments, R¹ and R² are each independently a radical of a keratolytic agent.

In some embodiments, each radical of a keratolytic agent described herein (e.g., Z, R¹, or R²) comprises one or more keratolytic group. In some embodiments, each radical of a keratolytic agent described herein (e.g., Z, R¹, or R²) comprises one or more group, each group being independently selected from the group consisting of —O—, oxo, substituted or unsubstituted (e.g., branched or straight) alkyl (alkylenyl), substituted or unsubstituted (e.g., branched or straight) heteroalkyl (heteroalkylenyl), substituted or unsubstituted alkoxyl, substituted or unsubstituted cycloalkyl, and substituted or unsubstituted heterocyclyl. In some embodiments, each radical of a keratolytic agent described herein (e.g., Z, R¹, or R²) is substituted or unsubstituted (e.g., branched or straight) alkyl (alkylenyl), substituted or unsubstituted (e.g., branched or straight) heteroalkyl (heteroalkylenyl), or substituted or unsubstituted heterocyclyl. In some embodiments, each radical of a keratolytic agent described herein (e.g., Z, R¹, or R²) is substituted or unsubstituted (e.g., branched or straight) alkyl (alkylenyl) or substituted or unsubstituted (e.g., branched or straight) heteroalkyl (heteroalkylenyl). In some embodiments, each radical of a keratolytic agent described herein (e.g., Z, R¹, or R²) is (e.g., branched or straight) alkyl (alkylenyl) substituted with one or more substituent, each substituent being independently selected from the group consisting of oxo and substituted or unsubstituted heterocyclyl (e.g., dithiolanyl or dithiolanyl oxide). In some embodiments, each radical of a keratolytic agent described herein (e.g., Z, R¹, or R²) is (e.g., branched or straight) heteroalkyl (heteroalkylenyl) substituted with one or more substituent, each substituent being independently selected from the group consisting of oxo, alkyl, thiol, thioalkyl, and substituted or unsubstituted heterocyclyl. In some embodiments, each radical of a keratolytic agent described herein (e.g., Z, R¹, or R²) is heterocyclyl substituted with one or more substituent, each substituent being independently selected from the group consisting of oxo, substituted or unsubstituted alkyl (e.g., alkyl substituted with oxo and thioalkyl), thiol, and thioalkyl. In some embodiments, each radical of a keratolytic agent described herein (e.g., Z, R¹, or R²) is branched alkyl (alkylenyl) substituted with one or more substituent, each substituent being independently selected from the group consisting of oxo, hydroxy, alkyl, and substituted or unsubstituted heterocyclyl. In some embodiments, each radical of a keratolytic agent described herein (e.g., Z, R¹, or R²) is straight alkyl (alkylenyl) substituted with one or more substituent, each substituent being independently selected from the group consisting of oxo, hydroxy, alkyl, and substituted or unsubstituted heterocyclyl. In some embodiments, each radical of a keratolytic agent described herein (e.g., Z, R¹, or R²) is branched heteroalkyl (heteroalkylenyl) substituted with one or more substituent, each substituent being independently selected from the group consisting of oxo, alkyl, thiol, thioalkyl, and substituted or unsubstituted heterocyclyl. In some embodiments, each radical of a keratolytic agent described herein (e.g., Z, R¹, or R²) is straight heteroalkyl (heteroalkylenyl) substituted with one or more substituent, each substituent being independently selected from the group consisting of oxo, alkyl, thiol, thioalkyl, and substituted or unsubstituted heterocyclyl.

In some embodiments, each radical of a keratolytic agent described herein (e.g., Z, R¹, or R²) comprises one or more keratolytic group. In some embodiments, each radical of a keratolytic agent described herein (e.g., Z, R¹, or R²) comprises one or more group, each group being independently selected from the group consisting of —O—, oxo, substituted or unsubstituted (e.g., branched or straight) alkyl (alkylenyl), substituted or unsubstituted (e.g., branched or straight) heteroalkyl (heteroalkylenyl), substituted or unsubstituted alkoxyl, substituted or unsubstituted cycloalkyl, and substituted or unsubstituted heterocyclyl. In some embodiments, each radical of a keratolytic agent described herein (e.g., Z, R¹, or R²) is (e.g., branched or straight) alkyl (alkylenyl) substituted with one or more substituent, each substituent being independently selected from the group consisting of oxo, hydroxy, alkyl, alkoxy, and substituted or unsubstituted heterocyclyl. In some embodiments, each radical of a keratolytic agent described herein (e.g., Z, R¹, or R²) is (e.g., branched or straight) heteroalkyl (heteroalkylenyl) substituted with one or more substituent, each substituent being independently selected from the group consisting of oxo, alkyl, thioalkyl, and substituted or unsubstituted heterocyclyl.

In some embodiments, each linker described herein (e.g., L, L¹, L², L^{A} or L^{B}) is independently a bond, substituted or unsubstituted (e.g., branched or straight) alkyl (alkylenyl), or substituted or unsubstituted (e.g., branched or straight) heteroalkyl (heteroalkylenyl). In some embodiments, each linker described herein (e.g., L, L¹, L², L^{A} or L^{B}) is independently substituted or unsubstituted (e.g., branched or straight) alkyl (alkylenyl) or substituted or unsubstituted (e.g., branched or straight) heteroalkyl (heteroalkylenyl). In some embodiments, each linker described herein (e.g., L, L¹, L², L^{A} or L^{B}) is independently a bond or substituted or unsubstituted (e.g., branched or straight) alkyl (alkylenyl). In some embodiments, each linker described herein (e.g., L, L¹, L², L^{A} or L^{B}) is independently a bond, >C(═O), —C(═O)O—, —C(═O)OCH(CH₃)—, —CH(CH₃)—, or —CH₂—. In some embodiments, each linker described herein (e.g., L, L¹, L², L^{A} or L^{B}) is independently a bond or >C(═O). In some embodiments, each linker described herein (e.g., L, L¹, L², L^{A} or L^{B}) is independently a bond, —CH(CH₃)—, or —CH₂—. In some embodiments, each linker described herein (e.g., L, L¹, L², L^{A} or L^{B}) is a bond. In some embodiments, the linker described herein (e.g., L, L¹, L², L^{A} or L^{B}) is >C(═O). the linker (e.g., L or L^{A}) is >C(═O).

In some embodiments, a linker described herein is a hydrolyzable linker.

In some embodiments, a linker described herein is a non-hydrolyzable linker.

In some embodiments, a compound described herein is stable in an aqueous environment. In some embodiments, the linker of a compound described herein is stable in an aqueous environment. In some embodiments, the aqueous environment is a buffered solution. In some embodiments, the aqueous environment is a biological environment. In some embodiments, the biological environment is skin. In some embodiments, the biological environment is in or around an eye.

In some embodiments, a compound described herein is enzymatically stable. In some embodiments, the linker of a compound described herein is enzymatically stable. In some embodiments, a compound described herein is not cleavable by enzymes. In some embodiments, the linker of a compound described herein is not cleavable by enzymes. In some embodiments, a compound described herein or the linker of a compound described herein is not cleavable by enzymes of the skin and/or eye. In some embodiments, a compound described herein or the linker of a compound described herein is not cleavable by enzymes of the skin. In some embodiments, a compound described herein or the linker of a compound described herein is not cleavable by enzymes of the eye. In some embodiments, the enzymes of the skin and/or eye are esterases, hydrolases, and/or reductases.

In some embodiments, the free form of a radical of a compound described herein (e.g., Y) is an antiparasitic agent. In some embodiments, a compound described herein has anti-parasitic activity. In some embodiments, a portion of a compound described herein has anti-parasitic activity. In some embodiments, a free form of a radical of a compound described herein has anti-parasitic activity. In some embodiments, the free form of the radical of a compound described herein is ivermectin. In some embodiments, the free form of the radical of a compound described herein (e.g., Y) is moxidectin.

In some embodiments, a compound described herein has antihistamine activity. In some embodiments, a portion of a compound described herein has antihistamine activity. In some embodiments, a free form of a radical of a compound described herein has antihistamine activity. In some embodiments, the free form of the radical of a compound described herein is an H1 antagonist, such as an HI antagonist described elsewhere herein.

In some embodiments, a compound described herein has keratolytic activity. In some embodiments, a portion of a compound described herein has keratolytic activity. In some embodiments, a free form of a radical of a compound described herein has keratolytic activity. In some embodiments, the free form of the radical of a compound described herein comprises a carboxylic acid, a thiol and/or a disulfide. In some embodiments, the free form of the radical of a compound described herein is or comprises lipoic acid, lipoic acid oxide, N-acetyl cysteine (NAC), captopril, and bucillamine.

In some embodiments, each radical of a keratolytic agent (e.g., Z, R¹, or R²) has a structure represented by:

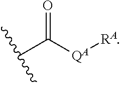

In some embodiments, Q^{A} is —O— or —(CR^{B}R^{C})ₘ—. In some embodiments, m is 1-6. In some embodiments, each R^{B} and R^{C} is independently H, halo, alkyl, alkoxy, haloalkyl, or thioalkyl. In some embodiments, an adjacent R^{B} and R^{C} combine to the atoms to which they are attached to form an oxo. In some embodiments, R^{A} is alkyl, heteroalkyl, heterocyclyl, alkoxy, or hydroxy, the alkyl, heteroalkyl, heterocyclyl, or alkoxy each independently being optionally substituted.

In some embodiments, Q^{A} is —O—.

In some embodiments, Q^{A} is —(CR^{B}R^{C})ₘ—.

In some embodiments, m is 1, 2, 3, 4, 5, or 6. In some embodiments, m is 1-4. In some embodiments, m is 1. In some embodiments, m is 2. In some embodiments, m is 3. In some embodiments, m is 4.

In some embodiments, each R^{B} and R^{C} is independently H, halo, alkyl, alkoxy, haloalkyl, or thioalkyl In some embodiments, each R^{B} and R^{C} is independently H or C₁-C₆ alkyl. In some embodiments, each R^{B} and R^{C} is independently H or CH₃. In some embodiments, an adjacent R^{B} and R^{C} combine to the atoms to which they are attached to form an oxo.

In some embodiments, R^{A} is substituted heteroalkyl. In some embodiments, R^{A} is substituted linear heteroalkyl. In some embodiments, R^{A} is substituted branched heteroalkyl. In some embodiments, R^{A} is substituted branched heteroalkyl substituted with one or more oxo and/or substituted or unsubstituted heterocyclyl. In some embodiments, R^{A} is substituted branched heteroalkyl substituted with one or more oxo and/or dithiolanyl or dithiolanyl oxide. In some embodiments, R^{A} is heteroalkyl substituted with oxo, alkyl, and/or thiol.

In some embodiments, R^{A} is substituted alkyl. In some embodiments, R^{A} is substituted linear alkyl. In some embodiments, R^{A} is substituted branched alkyl. In some embodiments, R^{A} is alkyl substituted with one or more oxo, hydroxy, and/or substituted or unsubstituted heterocyclyl.

In some embodiments, R^{A} is alkyl substituted with one or more oxo, hydroxy, and/or dithiolanyl or dithiolanyl oxide. In some embodiments, R^{A} is alkyl substituted with substituted or unsubstituted heterocyclyl. In some embodiments, R^{A} is alkyl substituted with substituted or unsubstituted dithiolanyl or dithiolanyl oxide.

In some embodiments, R^{A} is optionally substituted heterocyclyl (e.g., dithiolanyl or dithiolanyl oxide). In some embodiments, R^{A} is dithiolanyl or dithiolanyl oxide.

In some embodiments, R^{A} is substituted or unsubstituted heterocyclyl. In some embodiments, R^{A} is heterocyclyl substituted oxo and alkyl or hydrocyclyl substituted with alkyl substituted with oxo and thioalkyl.

In some embodiments, R^{A} comprises a thiol and/or a disulfide. In some embodiments, R^{A} comprises a thiol. In some embodiments, R^{A} comprises a disulfide. In some embodiments, R^{A} comprises a thiol and a disulfide.

In some embodiments, R^{A} is:

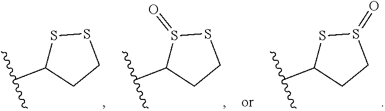

In some embodiments, Q^{A} is O and R^{A} is unsubstituted alkyl or substituted or unsubstituted heteroalkyl. In some embodiments, Q^{A} is O and R^{A} is heteroalkyl substituted with oxo and/or optionally substituted heterocyclyl. In some embodiments, Q^{A} is O and R^{A} is heteroalkyl substituted with oxo and/or dithiolanyl or dithiolanyl oxide.

In some embodiments, Q^{A} is —O— and R^{A} is optionally substituted C₁-C₆ alkyl.

In some embodiments, R^{A} is methyl, ethyl, propyl, isopropyl, butyl, or tert-butyl.

In some embodiments, Q^{A} is —(CR^{B}R^{C})ₘ—, m is 1-4, R^{B} and R^{C} are each independently H or C₁-C₆ alkyl, and R^{A} is optionally substituted heterocyclyl, alkyl substituted with optionally substituted heterocyclyl, alkyl substituted with one or more oxo and hydroxy, hetreoalkyl substituted with optionally substituted heterocyclyl, or heteroalkyl substituted with one or more oxo. In some embodiments, Q^{A} is —(CR^{B}R^{C})ₘ—, m is 1-4, R^{B} and R^{C} are each independently H or C₁-C₆ alkyl, and R^{A} is optionally substituted heterocyclyl. In some embodiments, Q^{A} is -(CR^{B}R^{C})ₘ—, m is 1-4, R^{B} and R^{C} are each independently H or C₁-C₆ alkyl, and R^{A} is alkyl substituted with alkyl substituted with one or more oxo and hydroxy. In some embodiments, Q^{A} is —(CR^{B}R^{C})ₘ—, m is 1-4, R^{B} and R^{C} are each independently H or C₁-C₆ alkyl, and R^{A} is hetreoalkyl substituted with optionally substituted heterocyclyl. In some embodiments, Q^{A} is —(CR^{B}R^{C})ₘ—, m is 1-4, R^{B} and R^{C} are each independently H or C₁-C₆ alkyl, and R^{A} is heteroalkyl substituted with one or more oxo.

In some embodiments, Q^{A} is —(CR^{B}R^{C})ₘ—, m is 1-4, R^{B} and R^{C} are each independently H or C₁-C₆ alkyl, and R^{A} is optionally substituted heterocyclyl.

In some embodiments, the optionally substituted heterocyclyl is dithiolanyl or dithiolanyl oxide.

In some embodiments, the optionally substituted heterocyclyl is:

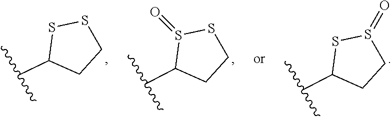 [SMILES: *C1CCSS1 ; *C1CCSS1=O ; *C1CCS(=O)S1]

In some embodiments, R^{A} is —CH₃, —CH(CH₃)₂, substituted C₁-C₆ alkyl, or substituted C₁-C₆ heteroalkyl. In some embodiments, R^{A} is —CH₃. In some embodiments, R^{A} is —CH(CH₃)₂. In some embodiments, R^{A} is substituted C₁-C₆ alkyl. In some embodiments, R^{A} is alkyl substituted with CH₃, oxo, hydroxy, and/or dithiolanyl or dithiolanyl oxide. In some embodiments, R^{A} is substituted C₁-C₆ heteroalkyl. In some embodiments, R^{A} is heteroalkyl substituted with CH₃, oxo, and/or dithiolanyl or dithiolanyl oxide.

In some embodiments, -Q-R^{A} is —CH₃, —CH(CH₃)₂, —OCH(CH₃)₂, —OCH(CH₃)OC(═O)CH(CH₃)₂, —CH₂CH₂C(═O)OCH(CH₃)OC(═O)OCH(CH₃)₂, —CH₂CH₂CH₂C(═O)OCH(CH₃)OC(═O)OCH(CH₃)₂, —CH₂CH₂C(═O)OH, —CH₂C(CH₃)₂C(═O)OH, —CH₂CH₂CH₂C(═O)OH, —C(═O)OCH(CH₃)OC(═O)OCH(CH₃)₂,

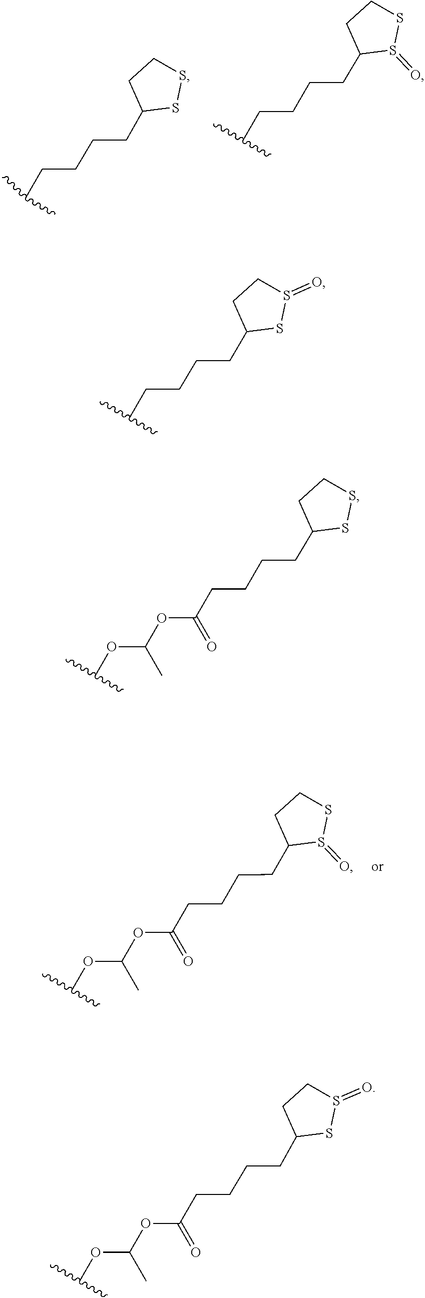 [SMILES: *CCCCC1CCSS1 ; *CCCCC1CCSS1=O ; *CCCCC1CCS(=O)S1 ; *OC(C)OC(=O)CCCCC1CCSS1 ; *OC(C)OC(=O)CCCCC1CCSS1=O ; *OC(C)OC(=O)CCCCC1CCS(=O)S1]

Each recitation of

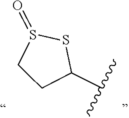 [SMILES: *C1CCS(=O)S1]

provided herein, unless otherwise stated, includes a specific and explicit recitation of:

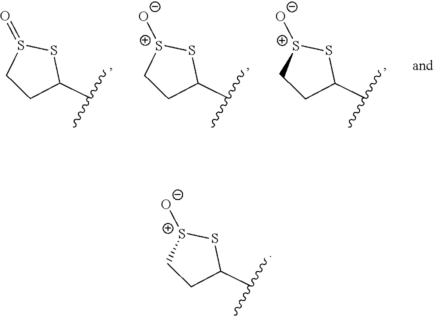

Each recitation of

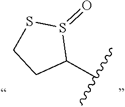 [SMILES: *C1CCSS1=O]

provided herein, unless otherwise stated, includes a specific and explicit recitation of:

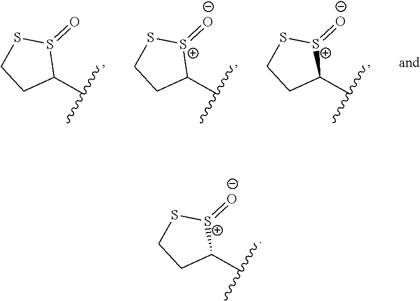

In some embodiments, a compound described herein has anti-parasitic activity. In some embodiments, a compound described herein hydrolyzes to provide moxidectin and a keratolytic agent (e.g., both moxidectin and the keratolytic having anti-parasitic activity). In some embodiments, a compound described herein hydrolyzes to provide moxidectin and a H1 antagonist (e.g., moxidectin having anti-parasitic activity and the H1 antagonist having anti-histamine and/or anti-inflammatory activity).

In some embodiments, a compound described herein has anti-parasitic activity. In some embodiments, a compound described herein hydrolyzes to provide ivermectin and a keratolytic agent (e.g., both ivermectin and the keratolytic having anti-parasitic activity). In some embodiments, a compound described herein hydrolyzes to provide ivermectin and a H1 antagonist (e.g., ivermectin having anti-parasitic activity and the H1 antagonist having anti-histamine and/or anti-inflammatory activity).

In some embodiments, a compound described herein is any compound described in International Publication Number WO 2024/134266, which is incorporated herein by reference in its entirety, in particular for the compounds and compositions provided therein as well as the methods and uses of the compounds and compositions provided therein. In some embodiments, the compound described herein comprises any linker described in International Publication Number WO 2024/134266, which is incorporated herein by reference in its entirety, in particular for the linkers provided therein. In some embodiments, the compound described herein comprises any anti-parasitic agent (or radical thereof) described in International Publication Number WO 2024/134266, which is incorporated herein by reference in its entirety, in particular for the anti-parasitic agents provided therein. In some embodiments, the compound described herein comprises any keratolytic agent described in International Publication Number WO 2024/134266, which is incorporated herein by reference in its entirety, in particular for the keratolytic agents provided therein. In some embodiments, the compound described herein comprises any HI antagonist (or radical thereof) described in International Publication Number WO 2024/134266, which is incorporated herein by reference in its entirety, in particular for the H1 antagonists provided therein.

In some embodiments, a compound provided herein is represented by any one of Formula (X), Formula (1-A), Formula (A-1), Formula (A-2), Formula (B-1), or Formula (B-2). In some embodiments, a compound provided herein is administered as a pure chemical. In other embodiments, a compound provided herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided in some embodiments herein is a pharmaceutical composition comprising at least one compound described herein together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

In some embodiments, a compound provided herein (e.g., a compound having a structure represented by any one of Formula (X), Formula (1-A), Formula (A-1), Formula (A-2), Formula (B-1), or Formula (B-2)) is substantially pure, in that it contains less than, for example, about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments provided herein is a pharmaceutical composition comprising a compound provided herein (e.g., a compound having a structure represented by any one of Formula (X), Formula (1-A), Formula (A-1), Formula (A-2), Formula (B-1), or Formula (B-2)) and at least one pharmaceutically acceptable excipient. In some embodiments, the pharmaceutical composition is suitable for dermal administration. In some embodiments, the pharmaceutical composition is suitable for ophthalmic administration. In some embodiments, the pharmaceutical composition is suitable for topical ophthalmic administration. In some embodiments, topical ophthalmic administration is administration in and/or around the eye, such as to the eyelid margin. In some embodiments, the pharmaceutical composition is suitable for administration to the eyelid margin. In some embodiments, topical ophthalmic administration is administration to the ocular surface and the inner surface to the eyelid.

Provided in some embodiments herein is a topical pharmaceutical composition (e.g., peridermal or periocular) comprising a compound having a structure described herein, such as a structure represented by Formula (X), Formula (1-A), Formula (A-1), Formula (A-2), Formula (B-1), or Formula (B-2).

In some embodiments, a compound described herein (e.g., a compound having a structure represented any one of Formula (X), Formula (1-A), Formula (A-1), Formula (A-2), Formula (B-1), or Formula (B-2)) is formulated as a solution or suspension for topical administration to the eye.

In some embodiments, a compound described herein (e.g., a compound having a structure represented any one of Formula (X), Formula (1-A), Formula (A-1), Formula (A-2), Formula (B-1), or Formula (B-2)) is formulated as a solution or suspension for topical administration to the skin.

Pharmaceutical compositions provided in some embodiments herein are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

In other embodiments, the topical compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier). Exemplary excipients are described, for example, in Remington: The Science and Practice of Pharmacy (Gennaro, 21st Ed. Mack Pub. Co., Easton, PA (2005)).

Provided in some embodiments herein is use of a compound having a structure described herein, such as a structure represented by Formula (X), Formula (1-A), Formula (A-1), Formula (A-2), Formula (B-1), or Formula (B-2), for treating a keratolytic disease or disorder in an individual. In some embodiments, the dermal or ocular disease or disorder is associated with keratin production. In some embodiments, the dermal or ocular disease or disorder is associated with keratinized material. In some embodiments, the dermal or ocular disease or disorder is associated with a build-up of keratinized material. In some embodiments, the individual has a bacterial or parasitic infection, such as a bacterial or parasitic infection described herein. In some embodiments, the individual has a demodex infestation or infection.

Provided in some embodiments herein is use of a compound having a structure described herein, such as a structure represented by Formula (X), Formula (1-A), Formula (A-1), Formula (A-2), Formula (B-1), or Formula (B-2), for treating a dermal or ocular disease or disorder in an individual. In some embodiments, the keratolytic disease or disorder is associated with keratinized material. In some embodiments, the keratolytic disease or disorder is associated with a build-up of keratinized material. In some embodiments, the individual has a bacterial or parasitic infection, such as a bacterial or parasitic infection described herein. In some embodiments, the individual has a demodex infestation or infection.

In some embodiments provided herein is a method of treating a dermatological or ophthalmic disease or disorder in a patient in need of thereof, comprising administering to the individual any compound provided herein, or a pharmaceutically acceptable salt thereof, or a (e.g., pharmaceutical) composition comprising any compound provided herein, or a pharmaceutically acceptable salt thereof, such as a compound represented by any structure herein, such as, for example, Formula (X), Formula (1-A), Formula (A-1), Formula (A-2), Formula (B-1), or Formula (B-2). In some embodiments provided herein the pharmaceutical composition is in the form of a solution or suspension suitable for (e.g., topical) ophthalmic administration. In some embodiments, (e.g., topical) ophthalmic administration is administration in and/or around the eye, such as to the eyelid margin. In some embodiments, (e.g., topical) ophthalmic administration is administration to the ocular surface and the inner surface to the eyelid. In some embodiments, the dermatological or ophthalmic disease or disorder is a microbial (e.g., demodex) infection or infestation, inflammation, and/or hyperkeratosis (e.g., of the eyes or skin). In some embodiments, the dermatological or ophthalmic disease or disorder is a microbial (e.g., demodex) infection or infestation, inflammation, and/or hyperkeratosis of the eyes or skin (e.g., the ocular surface). In some embodiments, the dermatological or ophthalmic dermatological disease or disorder is selected from the group consisting of meibomian gland dysfunction (MGD), (e.g., inflammatory and/or aqueous) dry eye disease (DED), blepharitis, seborrheic blepharitis, and an ocular allergy (e.g., a severe ocular allergy). In some embodiments, the dermatological or ophthalmic disease or disorder is inflammation or hyperkeratosis (e.g., of the eyes or skin).

In some embodiments, the dermal or the ocular disease or disorder is associated with keratosis, microbial infiltration, microbial infection, inflammation, or any combination thereof. In some embodiments, the dermal or the ocular disease or disorder is associated with keratosis. In some embodiments, the dermal or the ocular disease or disorder is associated with microbial infiltration. In some embodiments, the dermal or the ocular disease or disorder is associated with demodex infiltration. In some embodiments, the dermal or the ocular disease or disorder is associated with microbial infection. In some embodiments, the dermal or the ocular disease or disorder is associated with demodex infection. In some embodiments, the dermal or the ocular disease or disorder is associated with inflammation.

In some embodiments, the dermal or the ocular disease or disorder is or is associated with an infection. In some embodiments, the dermal or the ocular disease or disorder is an infection. In some embodiments, the dermal or the ocular disease or disorder is associated with an infection. In some embodiments, the disease or disorder is or is associated with a bacterial or parasitic infection. In some embodiments, the disease or disorder is a bacterial or parasitic infection. In some embodiments, the disease or disorder is associated with a bacterial or parasitic infection. In some embodiments, the disease or disorder is or is associated with a chlamydia infection. In some embodiments, the disease or disorder is or is associated with a protozoa, mite, worm, onchocerciasis, lice, scabies, nematode, or the like infection. In some embodiments, the disease or disorder is or is associated with a demodex infection. In some embodiments, the disease or disorder is a demodex infection. In some embodiments, the disease or disorder is associated with a demodex infection. In some embodiments, the disease or disorder is or is associated with demodex mites (e.g., Demodex Demodex Brevis, Demodex folicularum, and Demodex canis). In some embodiments, the disease or disorder is demodex mites (e.g., Demodex Brevis, Demodex folicularum, and Demodex canis). In some embodiments, the disease or disorder is associated with demodex mites (e.g., Demodex Brevis, Demodex folicularum, and Demodex canis). In some embodiments, the dermal or the ocular disease or disorder is a demodex infestation or infection. In some embodiments, the dermal or the ocular disease or disorder is associated with a demodex infestation or infection.

In some embodiments, the dermal or the ocular disease or disorder is an allergy or allergy symptom. In some embodiments, the dermal or the ocular disease or disorder is itching or hypersensitivity. In some embodiments, the dermal or the ocular disease or disorder is itching. In some embodiments, the dermal or the ocular disease or disorder is hypersensitivity.

In some embodiments, the ophthalmic disease or disorder is selected from the group consisting of dry eye, lid wiper epitheliopathy (LWE), contact lens discomfort (CLD), dry eye syndrome, evaporative dry eye syndrome, aqueous deficiency dry eye syndrome, blepharitis, keratitis, meibomian gland dysfunction, conjunctivitis, lacrimal gland disorder, contact lens related conditions and inflammation of the anterior surface of the eye, infection of the anterior surface of the eye, and autoimmune disorder of the anterior surface of the eye.

In certain embodiments, methods provided herein involve the method of treating meibomian gland dysfunction (MGD).

In some embodiments, the ocular disorder is a surface disorder, such as MGD, dry eye and associated inflammatory, bacterial, and parasitic disease, an (e.g., severe) ocular allergy), or a (e.g., inflammatory and/or aqueous) dry eye disease.

Provided herein is a method for treating an ocular surface disorder in an individual in need thereof comprising topical administration of a compound described herein to the individual in need thereof.

In some embodiments provided herein is a method for treating meibomian gland dysfunction in a patient in need thereof, comprising topically administering to the patient a composition comprising a therapeutically-effective amount of at least one compound described herein in an ophthalmically-acceptable carrier. In some embodiments, the topical administration of the composition comprising a therapeutically-effective amount of at least one compound described herein in an ophthalmically-acceptable carrier results in enhanced meibum production.

In some embodiments, the topical administration of the composition comprising a therapeutically-effective amount of at least one compound described herein in an ophthalmically-acceptable carrier occurs until the keratinized obstruction is relieved.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### I. Biological Evaluation

### Example 1: Rabbit Cornea Homogenate Stability Assay

Determining Rabbit Cornea Homogenate stability of the test compounds is performed using UPLC-MS. The assay is performed at two concentrations of Rabbit Cornea Homogenate (0.15 mg/mL and 0.45 mg/mL total protein) so that any hydrolysis observed can be assigned as esterase dependent or not.

### Rabbit Cornea Homogenisation

Three to four rabbit corneas (e.g., New Zealand Whites (NZW) or Dutch Belted (DB)) of approx. 50 mg each are sliced and scraped with a scalpel and tweezers until reduced to small (1-3 mm), thin pieces. These are transferred into a glass vial containing approximately 2 mL of cold DPBS pH 7.4 buffer.

Sample is cooled intermittently on ice and shear homogenized for 3 minutes, then centrifuged for 3 min at 13,000 g. The supernatant is pipetted off into a vial, and total protein concentration determined at 280 nm. Sample is stored at −78° C.

### Rabbit Cornea Esterase Assay

### Preparation of Stock Solutions:

10 mM Compound DMSO stocks are diluted to 10 μM in a glass vial: 10 μL of 10 mM Compound stock is added to 9,990 pl 50 mM DPBS, pH 7.4 buffer. Esterase homogenate is diluted to 300 ng/μl and 900 ng/μL in DPBS.

### Assay Conditions:

A heater shaker is set to 37° C. Into a suitable 96 well plate (Run Plate), 70 μL of 300 or 900 ng/μl esterase homogenate is pipetted into two rows as compounds are analyzed in duplicate (2 min, 5 min, 10 min, 20 min and 45 min). The plate is sealed and then warmed at 37° C. for 5 min.

Two 96 deep-well plates are put on ice (Kill Plates). To these, 990 μL of 50:50 MeCN—H₂O are added to required rows, labelled 0 min 2 min, 5 min, 10 min, 20 min and 45 min. The plates are covered to minimize evaporation.

To both rows of the Run Plate, 70 μL of 10 μM compound solution is added. At the appropriate time point, 10 μL of the assay mixture is added to the matching kill plate well containing 990 μL of 50:50 cold MeCN—H₂O. Samples are analyzed as soon as practicable by UPLC-MS (Waters Xevo TQ-S).

### Assay Conditions for Lipoic Acid Analysis:

A heater shaker is set to 37° C. Into a suitable 96 well plate (Run Plate), 80 μL of 300 or 900 ng/μL esterase homogenate is pipetted into two rows as compounds are analyzed in duplicate (2 min, 5 min, 10 min, 20 min and 45 min). The plate is sealed and then warmed at 37° C. for 5 min.

Two 96 shallow-well plates are placed on ice (Kill Plates). To these, 180 μL of 60:40 MeCN—H₂O+0.1% acetic acid are added to required rows. The plates are sealed to minimize evaporation.

To both rows of the Run Plate, 80 μL of 10 μM compound solution is added. At the appropriate time point, 20 μL of the assay mixture is added to the matching kill plate well containing 180 μL of 60:40 cold MeCN—H₂O+0.1% acetic acid. For lipoic acid analysis, samples are analyzed as soon as practicable by LCMS (Waters Xevo TQ-S). For parent conjugate and parent analysis the samples are diluted further 1 in 10:20 μL supernatant is added to 180 μL of 50:50 MeCN—H₂O.

Parent conjugate, parent and keratolytic concentrations are determined against appropriate standard response curves and the half-life (T½) of the parent conjugate is calculated using the measured concentration of the parent conjugate at each time point in the linear region of the log −linear plot.

### Example 2: Aqueous Hydrolysis Stability Assay

Determination of aqueous stability of the test compounds is performed using UPLC-MS. A test compound 10 mM stock solution is prepared in DMSO. 10 μL of the DMSO stock solution is dissolved in 990 μL of DPBS pH 7.4 buffer to prepare a 100 μM stock. A further dilution is made by dissolving 75 μl of 100 μM stock into 225 μL of DPBS. Final DMSO concentration is 0.25%. The solution is kept at 37° C. and injected without delay into the LCMS (Waters Xevo TQ-). Additional injections are performed at appropriate time points.

Half-life (T_{1/2}) of the parent conjugate is calculated using the peak area or measured concentration of the parent conjugate at each time point in the linear region of the log—linear plot.

### Example 3: Mouse Model of Experimental Dry Eye Disease

Female C57BL/6 mice (6-8 weeks old) or female HEL BCR Tg mice (6-8 weeks old) are commercially obtained. Experimental dry eye is induced as described by Niederkorn, et al. (J. Immunol. 2006,176:3950-3957) and Dursun et al. (Invest. Ophthalmol. Vis. Sci. 2002, 43:632-638). In brief, mice are exposed to desiccating stress in perforated cages with constant airflow from fans positioned on both sides and room humidity maintained at 30% to 35%. Injection of scopolamine hydrobromide (0.5 mg/0.2 mL; Sigma-Aldrich, St. Louis, MO) is administered subcutaneously, three times a day (8:00 AM, 12:00 noon, and 5:00 PM), on alternating hind-flanks to augment disease. Mice are exposed to desiccating stress for 3 weeks. Untreated control mice are maintained in a nonstressed environment at 50% to 75% relative humidity without exposure to forced air. Test animals are exposed to test compound and subsequently tear samples are obtained to determine stability of test compounds, and tissue samples are taken to determine presence of pro-inflammatory biomarkers.

### Example 4: Thiol Assay

### Stratum Corneum Preparation

Epidermis pieces are transferred and incubated overnight from 25-37° C. in a container containing 100 mL of 0.0005% trypsin (diluted in PBS). The stratum corneum pieces are removed and washed twice with HPLC grade water in a petri dish (145 mm), removing intact cells. The dish and/or pieces are shaken, producing nearly transparent layers. The stratum corneum is then transferred to a petri dish (145 mm), washed with hexane, and shaken to remove fats. Each piece is gently mounted on an absorbent paper. Each piece is transferred to an Eppendorf tube, allowing residual solvents to evaporate for a few minutes.

### Thiol Assay

Compounds (e.g., 50 μL; 1 μM to 800 μM) are applied to the isolated stratum corneum at room temperature for a period of 1-24 hours. The pieces are gently mixed with the compounds by pipetting. Following incubation, about 200 μL of 10 M sodium hydroxide is added, incubating for 1 hour at room temperature with continuous blending (e.g., vortexing) until the stratum corneum disintegrates. About 200 μL of 10 M hydrochloric acid is added to normalize pH, vortexing the samples. The samples are centrifuged (e.g., for 20 min at 16,000×g) at room temperature. The supernatant (middle layer) is transferred to an Eppendorf tube. The free thiols are isolated by adding tricholoracetic acid (e.g., 400 μL) and vortexing. The tubes are centrifuged (e.g., for 10 min at 16,000×g) at room temperature. The supernatant is removed, and Ellman's reagent solution (e.g., 220 μL) is added to the remaining pellet. After mixing, 100 μL for each tube is transferred to a 96 well plate in the dark. The plate is incubated for about 5 minutes at room temperature while shaking. The optical absorbance at 412 nm is detected and recorded.

We claim:

1. A compound having a structure represented by Formula (X): 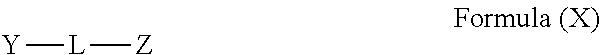
wherein:
Y has a structure represented by Formula (1-A): 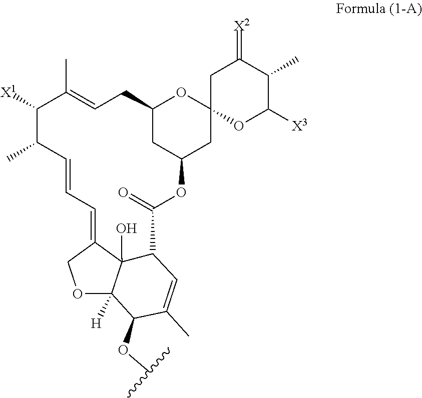
wherein,
X¹ is absent or; 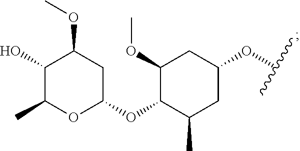
X² is absent or ═NOCH₃; and
X³ is substituted or unsubstituted alkyl;
Z is a radical of a keratolytic agent or a radical of an H1 antagonist;
L is a linker;
or a pharmaceutically acceptable salt or solvate thereof.

2. The compound of claim 2, wherein X¹ is absent.

3. The compound of claim 2, wherein X² is ═NOCH₃.

4. The compound of claim 2, wherein X³ is branched alkyl.

5. The compound of claim 5, wherein X³ is —C(CH₃)═CHCH(CH₃)₂.

6. The compound of claim 1, wherein Y is a radical of moxidectin.

7. The compound of claim 1, wherein Z is a radical of a keratolytic agent.

8. The compound of claim 8, wherein the radical of a keratolytic agent is substituted or unsubstituted alkyl or substituted or unsubstituted heteroalkyl.

9. The compound of claim 8, wherein the radical of a keratolytic agent is alkyl substituted with one or more substituent, each substituent being independently selected from the group consisting of oxo, hydroxy, alkyl, and substituted or unsubstituted heterocyclyl.

10. The compound of claim 8, wherein the radical of a keratolytic agent is heteroalkyl substituted with one or more substituent, each substituent being independently selected from the group consisting of oxo, alkyl, thiol, thioalkyl, and substituted or unsubstituted heterocyclyl.

11. The compound of claim 1, wherein the radical of a keratolytic agent has a structure represented by: 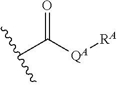
wherein:
Q^{A} is —O— or —(CR^{B}R^{C})ₘ;
m is 1-6;
each R^{B} and R^{C} is independently H, halo, alkyl, alkoxy, haloalkyl, or thioalkyl;
or an adjacent R^{B} and R^{C} combine to the atoms to which they are attached to form an oxo; and
R^{A} is alkyl, heteroalkyl, heterocyclyl, alkoxy, or hydroxy, the alkyl, heteroalkyl, heterocyclyl, or alkoxy each independently being optionally substituted.

12. The compound of claim 12, wherein each R^{B} and R^{C} is independently H or C₁-C₆ alkyl.

13. The compound of claim 12, wherein m is 1-4.

14. The compound of claim 12, wherein R^{A} is substituted heteroalkyl.

15. The compound of claim 12, wherein R^{A} is substituted alkyl.

16. The compound of claim 12, wherein R^{A} is substituted or unsubstituted heterocyclyl.

17. The compound of claim 12, wherein Q^{A} is O.

18. The compound of claim 1, wherein Z is a radical of an H₁ antagonist.

19. The compound of claim 18, wherein the H₁ antagonist is selected from the group consisting of olopatadine, cetirizine, acrivastine, bilastine, fexofenadine, levocabastine, hydroxyzine, periciazine, and quetiapine.

20. The compound of claim 1, wherein L is a bond or substituted or unsubstituted alkyl.
